(19) United States
(12) Patent Application Publication
(10) Pub. No.: US 2003/0049619 A1
(43) Pub. Date: Mar. 13, 2003

(54) Methods for the synthesis of polynucleotides and combinatorial libraries of polynucleotides

(76) Inventors: Simon Delagrave, Avondale, PA (US); Barry Marrs, Kennett Square, PA (US)

Correspondence Address: Christine A. Goddard, Ph.D., WOODCOCK WASHBURN KURTZ, MACKIEWICZ & NORRIS LLP, One Liberty Place - 46th Floor, Philadelphia, PA, 19103, US

(21) Appl. No.: 09/813,408

(22) Filed: Mar. 21, 2001

(51) Int. Cl. C12Q 1/68; C12P 19/34

(52) U.S. Cl. 435/6; 435/91.2

(57) ABSTRACT

Methods for the synthesis of polynucleotides and derivatives thereof are provided. Methods for the preparation of combinatorial libraries of polynucleotides are also provided. In addition, methods for the preparation and identification of polynucleotides having a predetermined property are provided.

### FIELD OF THE INVENTION

The present invention relates generally to methods for the synthesis of polynucleotides and derivatives thereof. The present invention also pertains to the preparation of combinatorial libraries of polynucleotides and the screening of libraries for polynucleotides having desirable properties.

### BACKGROUND OF THE INVENTION

The field of genomics is progressing at a tremendous rate. Not only has the human genome been sequenced, but so have the genomes of over thirty microbial species and animals such as the fruitfly D. melanogaster and the worm C. elegans. Experts predict that the complete sequences of more than one hundred additional microbial species will be available in the near future (Fraser, et al., Curr. Opin. Microbiol., 2000, 3, 443). This is in addition to the millions of gene sequences already available in public databases.

Accompanying the rapid progress in genomics is a growing interest in the field of directed evolution, as well as related areas of biotechnology, which are rapidly enabling the use of biomolecules (e.g., enzymes and DNA) for a variety of applications in medicine and chemistry (Chartrain, et al., Curr. Opin. Biotechnol., 2000, 11, 209 and Marrs, et al., Curr. Opin. Microbiol., 1999, 2, 241). There are early signs that biotechnology will even make significant contributions to computation and materials science (Mao, et al., Nature, 2000, 407, 493 and Whaley, et al., Nature, 2000, 405, 626). In order for scientists to fulfill the promise of biotechnology in these diverse areas, new methods of polynucleotide (e.g., genes, DNA, RNA) synthesis and, particularly, new methods of creating populations of polynucleotides from which useful variants can be isolated, will be highly desirable. Provided with such methods, scientists will be able to use more efficiently the enormous amounts of information contained in the genomic databases.

While it is possible to isolate genes and DNA molecules from almost any single organism found in nature, a method to efficiently synthesize such molecules in the laboratory is currently unavailable due to the sheer size and complexity of genetic material. Short polynucleotides (oligonucleotides) can be readily synthesized, but the methods for their synthesis are limited to stretches of up to about 100 bases. These methods are not capable of synthesizing polynucleotides on the order of about 1000 bases, the size range of a typical gene. A method for the synthesis of such large polynucleotides is desirable since it would allow genetic research to be conducted with greater precision and rapidity. For instance, when presented with a phylogeny of DNA sequences from a genomic database, a scientist may wish to compare and/or recombine these sequences to generate a population of molecules from which a useful variant (and/or recombinant) may be isolated using an appropriate screen or selection. In order to accomplish this task, a typical laboratory would need to isolate genes from a multitude of organisms and/or maintain a large collection of thousands of genes from hundreds of organisms, both daunting feats using present technology. While there have been attempts to commercialize such services, the profitability of these enterprises has not yet been demonstrated and the costs to their customers is, in many cases, prohibitive. Thus, a rapid and efficient method for the synthesis of large polynucleotides would greatly facilitate the manipulation of large amounts of genetic material.

Several methods of de novo polynucleotide synthesis have been described. For example, U.S. Ser. No. 09/571,774 describes the solid phase synthesis of polynucleotides by sequential ligation of oligonucleotide segments. In a somewhat different synthetic strategy, U.S. Pat. No. 5,942,609 and Chen, et al., Nucleic Acids Res., 1990, 18, 871 describe polynucleotide synthesis from preassembled oligonucleotides by hybridization of complementary bridging oligonucleotides. Preassembly by hybridization is associated with several disadvantages of this method. For instance, hybridization can pose problems related to the formation of unwanted secondary structure or non-specific hybridization. Hybridization also contributes to the labor intensiveness of the synthetic method, requiring “extra” oligonucleotides to be synthesized for each joint. Other polynucleotide synthetic methods are limited to the preparation of double-stranded polynucleotides. Examples of these methods are described in Ivanov, et al., Gene, 1990, 95, 295; Stahl, et al., Biotechniques, 1993, 14, 424; Hostomsky, et al., Nucleic Acids Symp. Ser., 1987, 18, 241; Hostomsky, et al., Nucleic Acids Res., 1987, 15, 4849; Beattie, et al., Nature, 1991, 352, 742; and Stemmer, et al., Gene, 1995, 164, 49.

As is evident from the methods described above, research in polynucleotide synthesis has typically concentrated on the coupling of oligonucleotides at double-stranded regions created by hydribization of complementary oligonucleotides. It is possible, however, to join polynucleotides, or oligonucleotides, without hybridization of complementary oligonucleotides. In fact, ligation of oligonucleotides without hybridization, using T4 RNA ligase, has been described (Walker, et al., Proc. Natl. Acad. Sci. USA, 1975, 72, 122 and Ohtsuka, et al., Nucleic Acids Res., 1976, 3, 1613), but was soon recognized to be problematic as a polynucleotide synthesis method due to the accumulation of unwanted byproducts (Krug, et al., Biochemistry, 1982, 21, 1858). Other characteristics of T4 RNA ligase reactions include long incubations and mediocre yields (Tessier, et al., Anal. Biochem., 1986, 158, 171). The synthesis of oligonucleotides from mononucleotide building blocks using T4 RNA ligase has recently been described (Schmitz, et al., Org. Lett., 1999, 1, 1729 and references therein) but is similarly plagued by long reaction times. Thus, the difficulties associated with this enzyme have deterred the development of single-stranded polynucleotide synthetic methods.

The enhanced ability for de novo synthesis of large polynucleotides or genes may greatly facilitate the preparation of combinatorial libraries of polynucleotides because it would be much more efficient than existing methods. For example, combinatorial libraries of genes can be made by cassette mutagenesis (Oliphant, et al., Gene, 1986, 44, 177 and Oliphant, et al., Proc. Natl. Acad. Sci. USA, 1989, 86, 9094) whereby genes with random combinations of nucleotides are created. Similarly, U.S. Pat. Nos. 5,723,323; 5,763,192; 5,814,476; and 5,817,483 describe libraries of expression vectors having stochastic DNA regions. By simultaneously randomly mutating fifteen nucleotides of a gene, a billion different sequences can be generated. Current methods of screening and molecular cloning often limit the number of sequences that can be screened to a much smaller number. Although there are examples of libraries with 10⁸ individual mutants (Cwirla, et al., Proc. Natl. Acad. Sci. USA, 1990, 87, 6378), certain screening methods to identify useful enzymes are limited to a few thousand mutants. A process to optimize combinatorial libraries has been proposed (Arkin, et al., Proc. Natl. Acad. Sci. USA, 1992, 89, 7811) and tested (Delagrave, et al., Protein Eng., 1993, 6, 327 and Delagrave, et al., Biotechnology, 1993, 11, 1548) to circumvent this problem. A related approach has also been proposed to deal with the combinatorial diversity of phylogenies of protein sequences (Goldman, et al., Biotechnology, 1992, 10, 1557). However, these methods consider only libraries having degeneracies at the nucleotide level. In some instances, such as for large sets of phylogenically related sequences, combinatorial libraries where degeneracies are at the oligonucleotide level (i.e., blocks of nucleotides), rather than at the nucleotide level, are more favorable. This difference would allow alteration of an entire sequence instead of at just a few nucleotides.

In an effort to prepare populations of polynucleotides, a method referred to as DNA shuffling has been developed. According to this method, described in U.S. Pat. No. 6,117,679 and Stemmer, et al., Proc. Natl. Acad. Sci. USA, 1994, 91, 10747, a series of related polynucleotides are isolated, fragmented, and recombined to form a population of polynucleotide variants. The recombination of related polynucleotides proceeds via hybridization of complementary or partially complementary fragments. The requirement for hybridization limits this method to polynucleotides with a certain minimal amount of homology. Moreover, recombination between polynucleotides tends to occur at points of high sequence identity which are found randomly along the sequences. There is, therefore, little control of the sites of recombination during a shuffling experiment. Furthermore, DNA shuffling methods are not amenable to working with RNA. However, in certain cases it may be advantageous to work directly with RNA molecules. For example, many viral genomes consist of single strands of RNA like flaviviruses such as Dengue, Japanese Encephalitis and West Nile, retroviruses such as HIV, and other animal and plant pathogens, including viroids (Fundamental Virology, Lippincott-Raven, Phildelphia, Pa., 1996) By constructing recombinant viral genomes, valuable vaccines may be developed (Guirakhoo, et al., Virology, 1999, 257, 363 and Monath, et al., Vaccine, 1999, 17, 1868), and the availability of methods to synthesize and recombine RNA more rapidly may accelerate this type of research.

De novo gene synthesis is a powerful technique that when fully optimized would contribute greatly to the fields of biotechnology and medicine. Not only would gene synthesis facilitate the manipulation of large polynucleotides by offering better control over, for example, the position of restriction sites, optimization of regions of sequence governing gene expression, and formation of chimeras; the ability to synthetically build a gene would allow the directed and rapid formation of combinatorial gene libraries. Screening of these libraries for genes with desired properties may allow the discovery or development of new and improved biomolecules such as enzymes with increased activity or receptors with higher ligand affinity. Thus, new methods for the synthesis of polynucleotides are needed, and the present invention is directed toward this need, as well as others.

### SUMMARY OF THE INVENTION

The present invention relates generally to the preparation of a polynucleotide having a target sequence from a plurality of oligonucleotides, wherein the sequences of the oligonucleotides comprise the target sequence of the polynucleotide, comprising coupling oligonucleotides of the plurality of oligonucleotides to form a plurality of coupled oligonucleotides, wherein each of the coupled oligonucleotides represents a region of the polynucleotide and shares at least one terminal region of sequence with at least one other coupled oligonucleotide, and assembling the polynucleotide by extension of the coupled oligonucleotides.

In some embodiments, the coupling of oligonucleotides is carried out by ligation with a ligase, preferably T4 RNA ligase. In further embodiments, at least one of the contiguous oligonucleotides undergoing coupling is attached to solid support. Furthermore, the resulting coupled oligonucleotide may also be attached to solid support. In other embodiments, at least one of the oligonucleotides undergoing coupling may be blocked at one end, and the blocking group may comprise or be capable of attaching to solid support. Preferably, coupled oligonucleotides comprise pairs of contiguous oligonucleotides, and assembly of the polynucleotide may be carried out by amplification using overlap PCR.

Other embodiments of the present invention are directed to methods of preparing a polynucleotide having a target sequence from a plurality of oligonucleotides, wherein the sequences of the oligonucleotides comprise the target sequence of the polynucleotide, comprising blocking the 3′ end of each of the oligonucleotides, except for the oligonucleotide comprising the 5′ terminus of said polynucleotide, with a blocking group to form a plurality of blocked oligonucleotides, coupling the 5′ end of each of the blocked oligonucleotides with the 3′ end of a further oligonucleotide of the plurality of oligonucleotides to form a plurality of coupled oligonucleotides, wherein the further oligonucleotide comprises a portion of the polynucleotide immediately 5′ to the sequence of the blocked oligonucleotides, wherein each of the coupled oligonucleotides shares at least one oligonucleotide with at least one other coupled oligonucleotide, and assembling the polynucleotide by extension of the coupled oligonucleotides.

Preferably, assembled polynucleotides comprise DNA, RNA, or DNA/RNA hybrids. Oligonucleotides may comprise from about 10 to about 200 nucleotides, and the blocking groups preferably comprise or are attached to solid support. Solid support may comprise agarose, polyacrylamide, magnetic beads, polystyrene, polyacrylate, controlled-pore glass, hydroxyethylmethacrylate, polyamide, polyethylene, polyethyleneoxy, and polyethyleneoxy/polystyrene copolymer. A preferred blocking group is ddUTP-biotin.

In some embodiments, coupling of oligonucleotides is carried out using a ligase. The coupling reaction is preferably a multi-step process comprising contacting a blocked oligonucleotide with ligase and cosubstrate to form activated oligonucleotide, washing the activated oligonucleotide to form washed oligonucleotide, and contacting the washed oligonucleotide with a further oligonucleotide and ligase. A preferred ligase is T4 RNA ligase and a preferred cosubstrate is ATP.

In some embodiments, coupled oligonucleotides are amplified prior to assembling the polynucleotide.

Other aspects of the invention include libraries of polynucleotides prepared by the methods described above.

In yet a further aspect, the present invention encompasses methods of coupling a first oligonucleotide with a further oligonucleotide, wherein the first oligonucleotide is attached to solid support, comprising contacting the first oligonucleotide with ligase and cosubstrate to form activated oligonucleotide, washing the activated oligonucleotide to form washed oligonucleotide, and contacting the washed oligonucleotide with the further oligonucleotide and ligase. Preferably, the oligonucleotides are single-stranded and the ligase is T4 RNA ligase. The cosubstrate is preferably ATP. Other substrates, known to those skilled in the art, can also be used.

The present invention also encompasses a method of preparing a library of polynucleotides from a plurality of oligonucleotides, wherein each of the polynucleotides shares a plurality of predetermined sequence positions occupied by the oligonucleotides, and wherein each of the polynucleotides comprises a different oligonucleotide in at least one predetermined sequence position, comprising coupling oligonucleotides of the plurality of oligonucleotides to form a plurality of coupled oligonucleotides wherein each of the coupled oligonucleotides shares at least one terminal region of sequence with at least one other coupled oligonucleotide, and assembling the polynucleotides by extension of the coupled oligonucleotides.

In some embodiments, the plurality of oligonucleotides is derived from a set of polynucleotides having at least one common property. The common property may be sequence homology, enzyme activity, or ligand binding. Preferably, the set of polynucleotides is optimized.

According to other embodiments, the present invention encompasses methods of preparing a library of polynucleotides from a plurality of oligonucleotides, wherein each of the polynucleotides share a plurality of predetermined sequence positions occupied by the oligonucleotides, and wherein each of the polynucleotides comprises a different oligonucleotide in at least one predetermined sequence position, comprising blocking the 3′ end of each of the oligonucleotides, except for the oligonucleotides comprising the 5′ terminus of the polynucleotides, with a blocking group to form a plurality of blocked oligonucleotides, coupling the 5′ end of each of the blocked oligonucleotides with the 3′ end of a further oligonucleotide of the plurality of oligonucleotides to form a plurality of coupled oligonucleotides, wherein the further oligonucleotide comprises a sequence position immediately 5′ to said sequence position of said blocked oligonucleotides, wherein each of the coupled oligonucleotides shares at least one oligonucleotide with at least one other coupled oligonucleotide, and assembling the polynucleotide by extension of the coupled oligonucleotides.

The present invention is further directed to methods of identifying a polynucleotide with a predetermined property, comprising generating a library of polynucleotides according to any of the methods described above, and selecting at least one polynucleotide within the library having the predetermined property.

Additionally, the present invention is directed to methods of identifying a polynucleotide with a predetermined property, comprising generating a library of polynucleotides according to any of the methods described above, selecting at least one polynucleotide within the library having the predetermined property; and repeating the library generation and polynucleotide selection wherein at least one oligonucleotide of the selected polynucleotides is preferentially incorporated into the library.

### BRIEF DESCRIPTION OF THE DRAWINGS

FIG. 1 outlines a representative embodiment for the preparation of a polynucleotide according to the methods of the present invention.

FIG. 2 outlines a representative embodiment for the preparation of a combinatorial library of polynucleotides according to the methods of the present invention.

FIG. 3 shows a phenogram of a phylogeny of 29 subtilisin-like amino acid sequences.

FIGS. 4A-4M show alignments of the 29 subtilisin-like amino acid sequences designated by accession numbers: SAgi119308 (SEQ ID NO: 1); gi267048 (SEQ ID NO: 2); SAgi730412 (SEQ ID NO: 3); SAgi6137335 (SEQ ID NO: 4); SAgi267046 (SEQ ID NO: 5); gi2970044 (SEQ ID NO: 6); gi2118104 (SEQ ID NO: 7); gi2118105 (SEQ ID NO: 8); gi11127680 (SEQ ID NO: 9); gi135016 (SEQ ID NO: 10); gi9837236 (SEQ ID NO: 11); gi995621 (SEQ ID NO: 12); gi995623 (SEQ ID NO: 13); gi995625 (SEQ ID NO: 14); gi9837238 (SEQ ID NO: 15); gi549004 (SEQ ID NO: 16); gi4139636 (SEQ ID NO: 17); gi230163 (SEQ ID NO: 18); gi135015 (SEQ ID NO: 19); gi773560 (SEQ ID NO: 20); gi494620 (SEQ ID NO: 21); gi494621 (SEQ ID NO: 22); gi2914658 (SEQ ID NO: 23); gi10173298 (SEQ ID NO: 24); gi2147106 (SEQ ID NO: 25); gi135010 (SEQ ID NO: 26); gi7435653 (SEQ ID NO: 27); gi10174108 (SEQ ID NO: 28); gi10173310 (SEQ ID NO: 29).

### DETAILED DESCRIPTION OF PREFERRED EMBODIMENTS

As used herein, the term “polynucleotide” means a polymer of nucleotides including ribonucleotides and deoxyribonucleotides, and modifications thereof, and combinations thereof. Preferred nucleotides include, but are not limited to, those comprising adenine (A), guanine (G), cytosine (C), thymine (T), and uracil (U). Modified nucleotides include, but are not limited to, those comprising 4-acetylcytidine, 5-(carboxyhydroxylmethyl)uridine, 2-O-methylcytidine, 5-carboxymethylaminomethyl-2-thiouridine, 5-carboxymethylaminomethyluridine, dihydrouridine, 2-O-methylpseudouridine, 2-O-methylguanosine, inosine, N6-isopentyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, 5-methoxyuridine, 5-methoxycarbonylmethyl-2-thiouridine, 5-methoxycarbonylmethyluridine, 2-methylthio-N6-isopentyladenosine, uridine-5-oxyacetic acid-methylester, uridine-5-oxyacetic acid, wybutoxosine, wybutosine, pseudouridine, queuosine, 2-thiocytidine, 5-methyl-2-thiouridine, 2-thiouridine, 4-thiouridine, 5-methyluridine, 2-O-methyl-5-methyluridine, 2-O-methyluridine, and the like. The polynucleotides of the invention can also comprise both ribonucleotides and deoxyribonucleotides in the same polynucleotide.

The phrase “target sequence,” as used herein, refers to a predetermined polynucleotide or corresponding amino acid sequence of one or more polynucleotides to be synthesized.

As used herein, the term “oligonucleotide” means a polymer of nucleotides, including ribonucleotides and deoxyribonucleotides, and modifications thereof, and combinations thereof, as described above, having up to about 200 bases. The polynucleotides of the present invention comprise a plurality of oligonucleotides. Oligonucleotides are polynucleotide building blocks, and each oligonucleotide occupies a unique “sequence position” in a polynucleotide that comprises it. Oligonucleotides having adjacent sequence positions are referred to as “contiguous.” Thus, assembly of contiguous oligonucleotides renders the polynucleotide to be synthesized.

The term “extension,” as used herein, means the growing of polynucleotides from oligonucleotides by, for example, sequential addition of mononucleotides to the oligonucleotide ends. The sequence to which mononucleotides are added is directed according to a template of predetermined sequence. In preferred embodiments, extension involves the polymerase chain reaction (PCR) in which polymerase catalyzes the addition of mononucleotides to oligonucleotide primers hybridized to a template. The resulting extension product is complementary to the template and may serve as primer for a further template sharing a terminal region of sequence with the original sequence template. In this way, polynucleotides can be generated from a plurality of shorter templates as long as the templates share terminal regions of sequence.

The term “degenerate,” as used herein, describes a sequence having a variable component. For instance, a polynucleotide that is degenerate at the oligonucleotide level comprises at least one sequence position that is occupied by different oligonucleotides.

The term “coupling,” as used herein, refers to the covalent joining of two molecules. In the case of coupling of oligonucleotides, coupling preferably refers to the covalent joining of oligonucleotides at their ends to form a linear “coupled oligonucleotide.”

As used herein, the term “contacting” means the bringing together of compounds to within distances that allow for intermolecular interactions and/or transformations. At least one “contacting” compound is preferably in the solution phase. Other “contacting” compounds may be attached to solid phase.

“Washing,” as used herein, refers to a step in a synthetic process that involves the removal of byproduct, excess reagent, solvent, buffer, any undesirable material, or any combination thereof, from a reaction product. Washing is facilitated when the reaction product is attached to solid phase and the unwanted material is in solution phase.

The term “library,” as used herein, refers to a plurality of polynucleotides or polypeptides in which substantially all the members have different sequences. “Combinatorial library” indicates a library prepared by combinatorial methods.

As used herein, the phrase “parent polynucleotides” or “parent set of polynucleotides” means a plurality of polynucleotides from which oligonucleotides are designed for the assembly of libraries.

As used herein, the phrase “oligonucleotide subset” or “subset of oligonucleotides” refers to a group of oligonucleotides within a plurality of oligonucleotides having a common sequence position. An “oligonucleotide subset” represents the oligonucleotides of a certain sequence position of a parent set of polynucleotides.

As used herein, the term “share” relates to items having the same characteristics. For instance, polynucleotides “share” regions of sequence when polynucleotides comprise substantially the same region of sequence. Additionally, polynucleotides that “share” properties have substantially the same properties.

As used herein, the term “homologous” or “homology” describes polynucleotide or polypeptides, or portions thereof, having a degree of sequence identity. Homology can be readily calculated by sequence comparisons using the BLAST computer program with default parameters.

As used herein, the term “screening” or “screen” refers to processes for assaying large numbers of library members for a “predetermined property” or desired characteristic. “Predetermined properties” include any distinguishing characteristic, such as structural or functional characteristics, of a polynucleotide or polypeptide including, but not limited to, primary structure, secondary structure, tertiary structure, encoded enzymatic activity, catalytic activity, stability, or ligand binding affinity. Some predetermined properties pertaining to enzyme and catalytic activity include higher or lower activities, broader or more specific activities, and activity with previously unknown or different substrates relative to wild type. Some predetermined properties related to ligand binding include, but are not limited to, weaker or stronger binding affinities, increased or decreased enantioselectivities, and higher or lower binding specificities relative to wild type. Other predetermined properties may be related to the stability of proteins, preferably enzymes, with respect to organic solvent systems, temperature, and sheer forces (i.e., stirring and ultrafiltration). Further, predetermined properties may be related to the ability of a protein to function under certain conditions related to temperature, pH, salinity, and the like. Predetermined properties are often the goal of directed evolution efforts in which a protein or nucleic acid is artificially evolved to exhibit new and/or improved properties relative to wild type.

As used herein, the phrase “ligand binding” refers to a property of a molecule that has binding affinity for a ligand. Ligands are typically small molecules such as, but not limited to, peptides, hormones, and drugs that bind to ligand-binding proteins such as, but not limited to, biological receptors, enzymes, antibodies, and the like.

The methods of the present invention are directed, inter alia, to the preparation of polynucleotides, libraries of polynucleotides, and polynucleotides having desired properties. Polynucleotides suitable for the present invention may include DNA, RNA, DNA/RNA hybrids, or derivatives thereof. The polynucleotide is preferably a gene, portion of a gene, a plasmid, cosmid, viral genome, bacterial genome, mammalian genome, origins of replication, or the like. Additionally, polynucleotides prepared by the present methods may be any length, but are preferably greater than about 100 nucleotides. More preferably, the polynucleotide comprises from about 400 nucleotides to about 100,000 nucleotides, more preferably from about 750 nucleotides to about 50,000 nucleotides, and even more preferably from about 1000 nucleotides to about 10,000 nucleotides.

In order to prepare a polynucleotide by the methods of the present invention, the sequence of the polynucleotide to be synthesized is preferably predetermined to facilitate its design and assembly. The predetermined sequence is simultaneously herein referred to as a target sequence, that could be, for example, the sequence of a gene. Methods for determining the sequence of a polynucleotide are well known to those skilled in the art and sequences are readily available in public databases such as GenBank. The polynucleotide can be thought of as composed of a finite number of smaller polynucleotides, or oligonucleotides, assembled in a certain order. The positions of each of the oligonucleotides within the polynucleotide are designated by sequence position. Since only a single order of oligonucleotides will yield the target sequence of the polynucleotide, each oligonucleotide has a unique sequence position. Oligonucleotides that have adjacent sequence positions are referred to as contiguous.

Oligonucleotides according to the present invention may be any length of no fewer than two nucleotides (nt) and no more than the length of the target sequence less two nucleotides. Preferably, oligonucleotides may range from about 10 to about 20 nt, from about 20 to about 30 nt, from 30 to about 50 nt, from about 50 to about 100 nt, or from about 100 to about 200 nt in length and may vary in size from each other. Oligonucleotides of any predetermined sequence comprising DNA and/or RNA are readily accessible, such as by synthesis on a commercially available nucleic acid synthesizer, and other methods for their syntheses and handling are well known to those skilled in the art.

Polynucleotides to be synthesized by the methods of the present invention are prepared by first coupling contiguous oligonucleotides end to end to form a plurality of coupled oligonucleotides of intermediate length (i.e., greater than the individual oligonucleotides undergoing coupling, but shorter than the full length polynucleotide). Each of the coupled oligonucleotides represents a region of the polynucleotide. The so formed plurality of coupled oligonucleotides is preferably designed such that all sequence positions of the desired polynucleotide are represented. The coupled oligonucleotides are further designed such that they share at least one terminal region of sequence with at least one other coupled oligonucleotide. Each coupled oligonucleotide comprises at least one region of sequence comprising a terminus (i.e., the terminal region of sequence) that is substantially identical with the terminal region of sequence of at least one other coupled oligonucleotide. For example, in a preferred embodiment, a first coupled oligonucleotide may be the result of coupling first and second oligonucleotides. Each of the first and second oligonucleotides of the first coupled oligonucleotide therefore includes a terminal region of sequence in the coupled oligonucleotide. Thus, a further coupled oligonucleotide, built from second and third oligonucleotides, would share terminal regions of sequence with the first coupled oligonucleotide because both coupled oligonucleotides comprise the same second oligonucleotide. In further embodiments, coupled oligonucleotides may comprise more than two oligonucleotides. For instance, three, four, five, six, or more oligonucleotides may be coupled to form coupled oligonucleotides. Coupled oligonucleotides having more than two oligonucleotides can be prepared, for example, by sequential coupling of the oligonucleotide components as described in U.S. Ser. No. 09/571,774, which is incorporated herein by reference in its entirety.

In preferred embodiments, the coupling of oligonucleotides proceeds in a fashion that results in covalent linkage of the oligonucleotides, preferably at their termini. Although any method of covalently linking oligonucleotides is suitable for the present invention, preferred embodiments may involve the ligation of oligonucleotides with a ligase. Ligation of DNA fragments using ligase is well known to those skilled in the art. A particularly preferred ligase is one that is capable of ligating single-stranded oligonucleotides such as an RNA ligase. T4 RNA ligase, or genetically modified versions thereof with enhanced catalytic activity, are particularly preferred RNA ligases. The coupling of oligonucleotides using T4 RNA ligase, and a method for obtaining a modified version of T4 RNA ligase, are described in detail in U.S. Ser. No. 09/571,774, incorporated herein by reference in its entirety. Alternatively, ribozymes may be used to ligate oligonucleotides.

Coupling of the oligonucleotides may be facilitated by using a blocking group and/or solid support attached to at least one of the oligonucleotides to be coupled. Blocking groups may aid in the assembly of oligonucleotides in the desired order and also may help prevent unwanted coupling reactions between non-contiguous oligonucleotides. In preferred embodiments, the 3′ end of one of the oligonucleotides to be coupled is blocked, thereby facilitating coupling of the unblocked 5′ end with the unblocked 3′ end of a further oligonucleotide. Blocking groups are well known to those skilled in the art and may include 3′ enzymatic acylation, a 3′ Pi group, and the like. Other suitable blocking groups and methods are described in Krug, et al., Biochemistry 1982, 21, 1858. Preferred blocking groups are capable of attaching to solid support or comprise solid support. A particularly preferred blocking group is ddUTP-biotin. This blocking group, which can be attached to the 3′ end of an oligonucleotide with deoxynucleotidyl transferase, substantially precludes ligation reactions at its site and allows binding of oligonucleotides to solid support. Blocking groups may be cleaved from oligonucleotides by reactions well known to those skilled in the art.

In some embodiments of the present invention, at least one of the contiguous oligonucleotides to be coupled is attached to solid support. Solid support facilitates manipulations in the assembly of the polynucleotide to be synthesized and is amenable to automation of the present methods. Solid support may also function as a blocking group. Any solid support may be suitable for the present invention so long as it does not substantially interfere with enzymatic reactions or bind non-specifically to polynucleotides or proteins. Suitable solid support may comprise agarose, polyacrylamide, magnetic beads, polystyrene, polyacrylate, controlled-pore glass, hydroxyethylmethacrylate, polyamide, polyethylene, polyethyleneoxy, or polyethyleneoxy/polystyrene copolymer, and the like. Oligonucleotides may be attached to and cleaved from solid support by methods well known to those skilled in the art. Examples of solid support and methods of immobilizing oligonucleotides thereto are described in, for example, U.S. Pat. No. 5,942,609, which is incorporated herein by reference in its entirety.

According to the methods of the present invention, the plurality of coupled oligonucleotides, comprising the oligonucleotides of the polynucleotide to be synthesized, are extended to assemble the full-length polynucleotide product. Preferably, extension is carried out by pooling and amplifying the plurality of coupled oligonucleotides, representing all sequence positions of the desired polynucleotide, together. Although amplification can be carried out by any means available, it is preferably carried out by the polymerase chain reaction (PCR) in the presence of appropriate primers. Preferred primers include an oligonucleotide that is substantially complementary to a region of sequence comprising the 3′ terminus of the target sequence and an oligonucleotide substantially identical with, or overlapping, the region of sequence comprising the 5′ terminus of the target sequence. In this fashion, the shared terminal regions of sequence in the plurality of coupled oligonucleotides serve as primers for extension. This type of PCR reaction is often referred to as overlap extension or overlap PCR and is well known to those skilled in the art. Overlap extension PCR methods involve the assembly of a polynucleotide from template segments. Generally, the segments comprise (or share) common regions of sequence at their termini that serve as primers for extension and assembly of the polynucleotide. References exemplifying the overlap PCR technique include Mullinax, et al., Biotechniques, 1992, 12, 864; Ye, et al., Biochem. Biophys. Res. Commun., 1992, 186, 143; Horton, et al., Gene 1989, 77, 61; and Ho, et al., Gene, 1989, 77, 51, each of which is incorporated herein by reference in its entirety.

In some embodiments, the polynucleotide is assembled directly by extension of coupled oligonucleotides attached to solid support. However, in other embodiments of the present invention, the coupled oligonucleotides may be individually amplified prior to assembling. Amplification can be carried out by any means, however, PCR amplification is preferable. Primers appropriate for PCR amplification of coupled oligonucleotides include oligonucleotides substantially complementary to the region of sequence comprising the 3′ end of each coupled oligonucleotide and oligonucleotides substantially identical to, or overlapping with, the 5′ end of each coupled oligonucleotide. Additionally, the 5′-most oligonucleotide of each coupled oligonucleotide may be used as primer.

Other amplification methods suitable for the present invention may include strand displacement amplification (Walker, et al., Proc. Natl. Acad. Sci. USA, 1992, 89, 392 and Walker, et al., Nucleic Acids Research, 1992, 20, 1691, each of which is incorporated herein by reference in its entirety), nucleic acid sequence based amplification (Compton, Nature, 1991, 350, 91 and Voisset, et al., Biotechniques, 2000, 29, 236, each of which is incorporated herein by reference in its entirety), and the like.

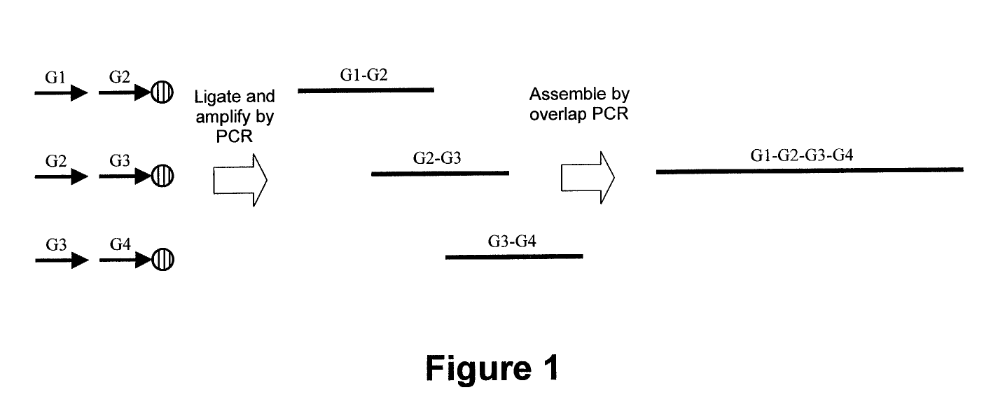

In some embodiments of the present invention, a polynucleotide may be assembled from a plurality of coupled oligonucleotides that each comprise pairs of contiguous oligonucleotides. As depicted in FIG. 1, the 3′ end (represented by an arrowhead) of each of the oligonucleotides, except for the oligonucleotide comprising the 5′ terminus of the target sequence, may be blocked with a blocking group (represented by a circle) to form a plurality of blocked oligonucleotides. Preferably, the blocking group comprises solid support or is further attached to solid support. The free 5′ end of each of the blocked oligonucleotides is then coupled with the 3′ end of a further oligonucleotide that comprises the portion of target sequence immediately 5′ to the sequence of the blocked oligonucleotides. Preferably, the further oligonucleotide is derived from the same set of oligonucleotides that were blocked. Each of the resulting coupled oligonucleotides of intermediate length, therefore, comprises two (or a pair of) contiguous oligonucleotides. The resulting set of coupled oligonucleotides contains each of the original oligonucleotides of the target polynucleotide, all of which are represented twice (i.e., once in two different coupled oligonucleotides), except for the oligonucleotides comprising the 3′ and 5′ ends of the target sequence which are represented once. It is in this fashion, for example, that the coupled oligonucleotides share terminal regions of sequence.

Using the plurality of coupled oligonucleotides as combined templates and primers, the target polynucleotide may be assembled by extension of the coupled oligonucleotides. During extension, coupled oligonucleotides may remain blocked, at their 3′ ends. According to preferred embodiments, the coupled oligonucleotides are pooled and amplified by overlap PCR in the presence of appropriate primers. Preferred primers include oligonucleotides complementary to the portion of target sequence comprising the 3′ end and oligonucleotides substantially identical with, or overlapping, the portion of target sequence comprising the 5′ end. Primer length can be any convenient length but typically range from about 5 nucleotides to about 30 nucleotides, or more preferably from about 15 nucleotides to about 25 nucleotides, or even more preferably from about 15 nucleotides to about 20 nucleotides. The target polynucleotide is thus formed by the extension of target sequence at overlapping regions of sequence in the set of coupled oligonucleotides.

In some instances, it may be desirable to individually amplify each of the coupled oligonucleotides prior to assembly. For instance, if yields of the coupling reaction are low, the coupled oligonucleotides may be amplified by PCR to yield material of sufficient quantitiy and/or purity to facilitate further manipulation. Coupled oligonucleotides may also be amplified by other amplification methods. Purification of amplified product may be carried out by gel electrophoresis and gel extraction as are well known to those skilled in the art. Amplification and electrophoresis techniques are exemplified in, for example, Sambrook, et al., (Eds.), Molecular Cloning: A Laboratory Guide, Cold Spring Harbor Laboratory Press: Cold Spring Harbor, N.Y. (1989), which is incorporated herein by reference in its entirety.

According to the methods of the present invention, the coupling of oligonucleotides is preferably carried out in the presence of a ligase. Ligases are well known to those skilled in the art as enzymes that are capable of ligating the blunt ends of nucleic acids. While not wishing to be bound by theory, it is believed that ligases catalyze the formation of a phosphodiester bond between the 3′-OH group at the end of one nucleic acid and the 5′-phosphate group at the end of another nucleic acid. The mechanism is believed to proceed through a nucleic acid-adenylate intermediate in which an AMP group is attached to the phosphate group at the 5′ terminus of a nucleic acid. The activated phosphate group then undergoes nucleophilic attack by the 3′-OH of a further nucleic acid, yielding the coupled nucleic acid. DNA ligases are specific for double-stranded nucleic acids, and their use as ligating reagents is well known to those skilled in the art. In contrast with DNA ligases, RNA ligases are capable of ligating single-stranded nucleic acids.

In view of the proposed ligation mechanism, methods for coupling oligonucleotides of the present invention comprise several steps. A first step involves contacting a first oligonucleotide with a ligase and cosubstrate to form an intermediate activated oligonucleotide. For oligonucleotides that are single-stranded, a preferred ligase is an RNA ligase, such as T4 RNA ligase. Cosubstrates can include ATP, NAD+, or other molecules depending on the specificity of the ligase. For instance, ATP cosubstrate is preferably used with T4 RNA ligase. In some embodiments, the first oligonucleotide is attached to a blocking group, preferably at the 3′ end. Alternatively, the blocking group comprises solid support or is attached to solid support to facilitate subsequent manipulations. The activated oligonucleotide is then washed to isolate it from residual reagents or byproducts. Not wishing to be bound by theory, it is thought that the activated oligonucleotide corresponds to an adenylated intermediate (when cosubstrate is ATP) which may be susceptible to nucleophilic attack by AMP byproducts. This side reaction may result in insertions of A or poly-A as well as contribute to poor yields of the desired coupled oligonucleotide. The washed oligonucleotide is then contacted with a further oligonucleotide and ligase to form the desired coupled oligonucleotide. Preferably, the further oligonucleotide comprises a free 3′-OH group. The contacting of washed oligonucleotide is preferably performed in the absence of any competing ligase substrates or cosubstrates including, but not limited to, ATP and AMP, or other reactants that may interfere with direct coupling of oligonucleotides. The resulting coupled oligonucleotide may be purified by subsequent washing and/or amplification.

Methods of the present invention include the preparation of libraries of polynucleotides. In general, libraries of polynucleotides comprise a plurality of different polynucleotides, typically generated by randomization or combinatorial methods, that may be screened for members having desirable properties. Libraries can comprise a minimum of two members but typically, and desirably, contain a much larger number. Larger libraries are more likely to have members with desirable properties, however, current screening methods have difficulty handling very large libraries (i.e., of more than a few thousand unique members). Thus, preferred libraries comprise from about 10¹ to about 10¹⁰, or more preferably from about 10² to about 10⁵, or even more preferably from about 10³ to about 10⁴ unique polynucleotide members.

Libraries of the present invention are characterized as a set, or plurality, of polynucleotides that share a plurality of predetermined sequence positions. These sequence positions serve as markers along the target sequences that indicate the desired order and position of each assembled oligonucleotide. Thus, each of the sequence positions are preferably occupied by an oligonucleotide. Furthermore, each of the polynucleotides of the library preferably comprises a different oligonucleotide in at least one sequence position. Different oligonucleotides differ by sequence. Different oligonucleotides may be of variable size, comprising insertions or deletions. Additionally, different oligonucleotides may constitute a set of degenerate oligonucleotides, varying at one or more nucleotide sites. Thus, individual polynucleotide members of the libraries differ in sequence from each other because their oligonucleotide compositions are different.

Libraries of the present invention are built up from a plurality of oligonucleotides. The plurality of oligonucleotides is composed of subsets of oligonucleotides, each subset corresponding to a certain sequence position. Subsets may contain a single oligonucleotide or any number of different oligonucleotides. At least one subset is comprised of more than one oligonucleotide. Upon synthesis, each polynucleotide member of the library is preferably assembled using one oligonucleotide per sequence position. For instance, if one oligonucleotide subset contains two different oligonucleotides, and the others contain only one oligonucleotide, then a library of two different polynucleotides can be assembled. The two library members differ by incorporation of different oligonucleotides at a certain sequence position. Thus, it is readily apparent that large combinatorial libraries can be generated from multiple oligonucleotide subsets having a plurality of different oligonucleotide members.

Oligonucleotides for assembling a library of polynucleotides can be selected in any number of ways. In some embodiments, oligonucleotides are constituents of a set of parent polynucleotides. The set of parent polynucleotides may comprise polynucleotides sharing any level of homology, including, for instance, little or no homology ranging from about 0% to about 10%, or about 10% to about 20%, or about 20% to about 30%, or about 30% to about 40%, or about 40% to about 50% identity at the nucleotide level. In some embodiments, the parent set of polynucleotides may share some homology at the amino acid level (e.g., greater than about 50% identity), yet share little or no homology at the polynucleotide level.

In other embodiments, the parent polynucleotides are related and share a common property, at the nucleotide or amino acid level, such as a physical characteristic or specific function. Although not a necessary condition as indicated above, the parent polynucleotides may be related by the physical characteristic of homology. For instance, related polynucleotides may possess homology at the nucleotide or amino acid level. Furthermore, homology may occur at the sequence level (such as primary structure), secondary structure level (such as, but not limited to, helices, beta-strands, hairpins, etc.), or tertiary structure level (such as, but not limited to, Rossman folds, beta-barrels, immunoglobin folds, etc.). Although, any level of homology (at either the nucleotide or amino acid level) may be used as a criterion for selecting a set of polynucleotides, preferred ranges of homology include, but are not limited to, at least about 50%, at least about 60%, at least about 70%, at least about 80%, at least about 90%, at least about 95%, or at least about 99% identity at the amino acid level. Other common properties suitable for selection of a set of polynucleotides include enzyme activity and ligand binding properties for the polynucleotides themselves or their expression products. For instance, sets of parent polynucleotides may comprise polynucleotides coding for particular enzymes that catalyze a desired chemical reaction or receptors that bind certain ligands.

In preferred embodiments, the set of parent polynucleotides can be selected according to their function. As a non-limiting example, one or more polynucleotide sequences may be identified from public sources, such as literature databases like PubMed, sequence databases like GenBank, or enzyme databases available on-line from ExPASy of the Swiss Institute of Bioinformatics, based on their ability to code for proteins capable of catalyzing a certain chemical reaction. Upon identification of a polynucleotide, others sharing homology at the nucleotide or amino acid level can be further identified using homology searching tools, such as BLAST (publically available online at www.ncbi.nlm.nih.gov/BLAST/).

Sets of parent polynucleotides may comprise any number of unique polynucletide sequences, however, it is often desirable to seek a balance between the preparation of large libraries that may potentially harbor an optimal variant and smaller libraries that are more easily managed and manipulated. For instance, a selected set of fewer than five parent polynucleotides can yield up to about 10⁵ different recombined sequences which provides diversity and is readily handled during screening. It is therefore apparent that polynucletide sets of five or more can readily result in exponentially larger libraries that are difficult to work with, are not amenable to present screening techniques, and may incur significant cost. Thus, it is often desirable to prepare an optimized set of polynucleotides that balances the needs for diverse libraries, easy manipulation, and low cost.

Optimization of parent polynucleotide sets can be achieved by a variety of methods. For example, an optimized set of parent polynucleotides can be selected from a larger set of polynucletides. The basis for selection can be a specific property, function, or physical characteristic that is desirable in the recombined sequences of the library. For instance, if a recombined polynucleotide sequence capable of coding for an enzyme that catalyzes a reaction at high pH is desired, then of the possible polynucleotide sequences that catalyze the reaction, only the ones that perform at high pH are selected to comprise the optimized set of polynucleotides. In another approach to making optimized sets of polynucleotides that makes fewer assumptions about the contribution of sequence to phenotype and allows for greater diversity, members of the optimized set may be chosen according to phylogenies. For example, a set of polynucleotides sharing a predetermined minimal sequence homology may be organized into a phylogenetic tree. Algorithms enabling the assembly of homologous sequences into phylogenetic trees are well known to those skilled in the art. For instance, the phylogenetic tree building program package Phylip is readily available to the public on-line at evolution.genetics.washington.edu/phylip.html maintained by the University of Washington. Sequences representing different branches of the calculated phylogenetic tree may then be selected to comprise an optimized set of polynucleotides.

The set of parent polynucleotides is dissected into oligonucleotides. Oligonucleotides may be chosen randomly or based on particular features of the polynucleotides. Oligonucleotides may also be chosen in order to facilitate their coupling. For example, it may be preferable for the 5′ terminus of oligonucleotides to be a C, rather than a G, because the enzyme T4 RNA ligase ligates acceptor oligonucleotides to a 5° C. more efficiently. In the event the parent polynucleotides share homology, the sequences may be aligned to facilitate identification of regions of sequence appropriate to represent a subset of oligonucleotides. For instance, a highly variable or highly conserved region of sequence may be designated to represent a subset of oligonucleotides. Sequence alignments are readily performed by those skilled in the art. An example of a suitable sequence alignment program is ClustalW v. 1.7, available online at clustalw.genome.adjp. Oligonucleotides may also be designed according to size. For example, subsets having longer oligonucleotides may result in libraries with less complexity than libraries comprising shorter oligonucleotides. Furthermore, oligonucleotides not directly derived from the selected polynucleotide set can be introduced into the library. For instance, certain mutations or degeneracies desired in the resulting library may be incorporated by adding oligonucleotides to the desired subsets (or sequence positions). Thus, great control can be maintained in engineering particular features into the library such as, but not limited to, restriction sites, point mutations, frame shifts, insertions, deletions, and the like.

In preferred embodiments, oligonucleotide subsets may be determined from their corresponding amino acid sequence subsets. Accordingly, in order to encode two or more amino acids at the same position in the same sequence (degeneracies), the following methods may be used. Most simply, it may be readily determined upon inspection that a basepair in one oligonucleotide differs from the analogous basepair of a further oligonucleotide, and the difference directly corresponds to a difference in one amino acid. Alternatively, a further embodiment involves determining oligonucleotide subsets from the amino acid sequences themselves. This approach may be facilitated using the computer program CyberDope which is available online at www.kairos-scientific.com/searchable/cyberdope.html and is described in Delagrave, et al., Protein Eng., 1993, supra., Delagrave, et al., Biotechnology 1993 supra, and Goldman, et al., supra. According to this program, a set of amino acids, for instance occupying a variable amino acid site in a set of polypeptides, may be entered, (e.g., A and S, or A, S and T). Based on the amino acids entered, the program calculates a set of degenerate codons. In alternative embodiments, the codon preferences (codon usage) of the host organism which will express the library of polynucleotides, may be taken into account when designing the oligonucleotides to avoid introducing disfavored codons.

If antisense complementary oligonucleotides are required in the preparation of the libraries (i.e., during amplification), care should be taken to maintain the degeneracies encoded in the above sense oligonucleotides. The use of inosine as a base complementary to a degenerate position has been described in the past (Reidhaar-Olson, et al., Science, 1988, 241, 53).

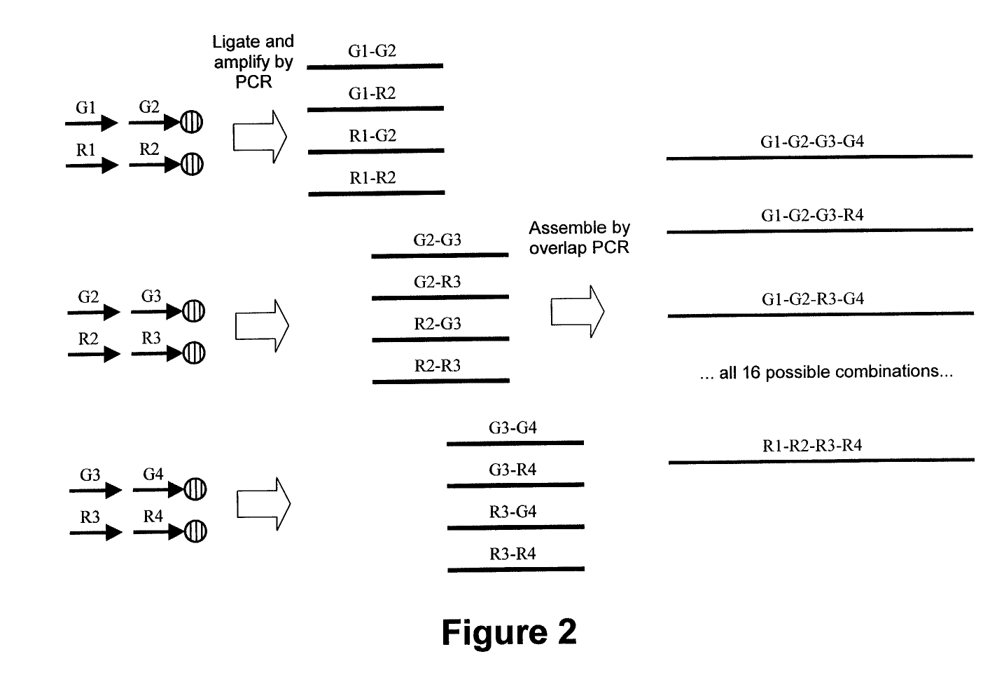

Libraries according to the present invention can be prepared from oligonucleotides by the procedures hereinbefore described. A representative example, FIG. 2 shows a method for the recombination of a set of two parent polynucleotide sequences (G and R), having four sequence positions numbered 1 to 4, each sequence position representing an oligonucleotide subset (e.g., G2 and R2), to generate a library of all 16 possible combinations. Generally speaking for the preparation of libraries, contiguous oligonucleotides, having adjacent sequence positions, are coupled to form coupled oligonucleotides that share terminal regions of sequence. Because one or more sequence positions can be represented by more than one oligonucleotide, coupled oligonucleotides preferably represent at least some, if not all, of the possible contiguous oligonucleotide combinations. For example, in FIG. 2, three groups of four different coupled oligonucleotide combinations are represented, where coupled oligonucleotides comprise two contiguous oligonucleotides. These groups are distinguished from each other by the sequence positions they represent. For illustrative purposes, FIG. 2 shows one coupled oligonucleotide group representing sequence positions 1 and 2, another group representing sequence positions 2 and 3, and a further group representing sequence positions 3 and 4. Each library member, according to the embodiment shown in FIG. 2, is a assembled from three coupled oligonucleotides, one from each group.

The library is assembled by extension of the coupled oligonucleotides. Preferably, the coupled oligonucleotides are pooled and amplified by PCR as herein described previously. Suitable primers for PCR amplification can be readily determined by one skilled in the art. Preferably, primers may include oligonucleotides complementary to regions of sequence comprising the 3′ termini of the target sequences of the library. For instance, in FIG. 2, suitable primers would be complementary to the 3′ end of oligonucleotides R4 and G4. Other preferred primers include oligonucleotides substantially identical with, or overlapping, regions of sequence comprising the 5′ termini of the target sequences of the library. In FIG. 2, for example, suitable primers include oligonucleotides G1 and R1, or portions thereof comprising the 5′ end.

Once generated, libraries of polynucleotides may be manipulated directly, or may be inserted into appropriate cloning vectors and expressed. Methods for cloning and expression of polynucleotides, as well as libraries of polynucleotides, are well known to those skilled in the art.

Libraries of polynucleotides, or the expression products thereof, may be screened for members having desirable new and/or improved properties. Any screening method that may result in the identification or selection of one or more library members having a predetermined property or desirable characteristic is suitable for the present invention. Methods of screening are well known to those skilled in the art and include, for example, enzyme activity assays, biological assays, or binding assays. Preferred screening methods include phage display and other methods of affinity selection, including those applied directly to polynucleotides. Other preferred methods of screening involve, for example, imaging technology and calorimetric assays. Suitable screening methods are further described in Marrs, et al., supra.; Bylina, et al., ASM News, 2000, 66, 211; Joyce, G. F., Gene, 1989, 82, 83; Robertson, et al., Nature, 1990, 344, 467; Chen, et al., Proc. Natl. Acad. Sci. USA, 1993, 90, 5618; Chen, et al., Biotechnology, 1991, 9, 1073; Joo, et al., Chem. Biol., 1999, 6, 699; Joo, et al., Nature, 1999, 399, 670; Miyazaki, et al., J. Mol. Evol., 1999, 49, 716; You, et al., Prot. Eng., 1996, 9, 77; and U.S. Pat. Nos. 5,914,245 and 6,117,679, each of which is incorporated herein by reference in its entirety.

Polynucleotides identified by screening of a library may be readily isolated and characterized. Preferably, characterization includes sequencing of the identified polynucleotides using standard methods known to those skilled in the art. Alternatively, sequencing and characterization may also be carried out using microarray technology. For instance, the same oligonucleotides used to assemble the library may be arrayed, such as in a “DNA chip,” and then probed using a labeled version (e.g., fluorescently tagged PCR product or transcript) of the polynucleotide to be sequenced. Microarray technology is described in, for instance, Southern, et al., Nat. Genet. 1999, 21, 5, which is herein incorporated by reference in its entirety.

In preferred embodiments of the present invention, a recursive screening method may be employed for preparing or identifying a polynucleotide with a predetermined property from a library. An example of a recursive screening method is recursive ensemble mutagenesis described in Arkin, et al., Proc. Natl. Acad. Sci. USA, 1992, 89, 7811; Delagrave, et al., Protein Eng., 1993, 6, 327; and Delagrave, et al., Biotechnology, 1993, 11, 1548, each of which is herein incorporated by reference in its entirety. According to this method, one or more polynucleotides, having a predetermined property, are identified from a first library by a suitable screening method. The identified polynucletides are characterized and the resulting information used to assemble a further library. For instance, one or more oligonucleotides of the identified polynucleotides may be preferentially incorporated into a further library which may also be screened for polynucleotides with a desirable property. Generating a library by incorporating the oligonucleotides identified from a previous cycle can be repeated as many time as desired. Preferably, the recursion is terminated upon identification of one or more library members having a predetermined or desirable property that is superior to the desirable property of the identified polynucleotides of previous cycles or that meets a certain threshold or criterion. According to this method, oligonucleotides that do not lead to functional sequences are eliminated from the pool of oligonucleotides used to generate the next library generation. Furthermore, amounts of oligonucleotides used in the preparation of a further library can be weighted according to their frequency of occurrence in the identified polynucleotides. Alternatively, if the identified polynucleotides are too small in number to accurately represent the true frequency of occurrence in a population of desirable polynucleotides, their amounts can be equally weighted. As an example, if the initial set of polynucleotides was chosen based on equal representation of branches of a phylogenetic tree, it is possible that certain families would be represented more frequently than others in the polynucleotides identified with a screen. Thus, polynucleotides belonging to these preferred families but not used in the initial generation of a library may be used to prepare a further library generation, thus expanding diversity while preserving a bias towards desirable sequences.

Collectively, the methods of the present invention allow for rapid and controlled “directed evolution” of genes and proteins. The present methods facilitate the preparation of biomolecules having desirable properties that are not naturally known or available. Uses for these improved biomolecules are widespread, promising contributions to the areas of chemistry, biotechnology, and medicine. Enzymes having improved catalytic activities and receptors having modified ligand binding affinities, to name a few, are just some of the possible achievements of the present invention.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated by reference in their entireties.

Examples 1-3 are actual while the remaining Examples are prophetic.

### EXAMPLES

### Example 1

### Preparation of a Single Polynucleotide

Oligonucleotides

The following oligonucleotides were synthesized by Operon Inc. (Alameda, Calif.):

G1(100 mer):

G1 (100mer):

| | |
|---|---|
| AGAGGATCCCCGGGTACCGGTAGAAAAAATGAGTA | (SEQ ID NO:30) |
| AAGGAGAAGAACTTTTCACTGGAGTTGTCCCAATT | |
| CTTGTTGAATTAGATGGTGATGTTAATGGG | |

G2 (60 mer, 5′ phosphorylated):

G2 (60mer, 5′ phosphorylated):

| | |
|---|---|
| CACAAATTTTCTGTCAGTGGAGAGGGTGAAGGTGA | (SEQ ID NO:31) |
| TGCAACATACGGAAAACTTACCCTT | |

G3 (60 mer, 5′ phosphorylated):

G3 (60mer, 5′ phosphorylated):

| | |
|---|---|
| AAATTTATTTGCACTACTGGAAAACTACCGGTTCC | (SEQ ID NO:32) |
| ATGGCCAACACTTGTCACTACTTTC | |

G4 (100 mer, 5′ phosphorylated):

| | |
|---|---|
| G4 (100mer, 5′ phosphorylated): | |
| TCTTATGGTGTTCAATGCTTTTCAAGATACCCAGA | (SEQ ID NO:33) |
| TCATATGAAACGGCATGACTTTTTCAAGAGTGCCA | |
| TGCCCGAAGGTTATGTACAGGAAAGAACTA | |
| pcrG1 (20mer): | |
| AGAGGATCCCCGGGTACCGG | (SEQ ID NO:34) |
| G2- (20mer): | |
| AAGGGTAAGTTTTCCGTATG | (SEQ ID NO:35) |
| G3- (20mer): | |
| GAAAGTAGTGACAAGTGTTG | (SEQ ID NO:36) |
| G4- (20mer): | |
| TAGTTCTTTCCTGTACATAA | (SEQ ID NO:37) |

All oligonucleotides were received purified by HPLC or PAGE, lyophilized and quantitated by the manufacturer. Once assembled in the correct order, 5′-G1-G2-G3-G4-3′, the resulting 320 bp-long polynucleotide encoded almost the entire 5′ half of the green fluorescent protein (GFP) gene.

Loading beads with oligos

Oligonucleotides were resuspended in water to yield 25 μM solutions, and ddUTP-biotin labeling of G2, G3 and G4 was performed by mixing in 3 separate tubes: 4 μL 25 μM of oligo G2, G3 or G4; 4 μL 5× buffer provided with enzyme; 4 μL CoCl₂ 25 mM; 1 μL 100 μM ddUTP-biotin (biotin-ε-aminocaproyl-γ-aminobutyryl-[5-(3aminoallyl)-2′,3′-dideoxyuridine-5′-triphosphate, Roche Molecular biochemicals, Mannheim, Germany); 1 μL terminal transferase (50 U/mL, Roche Molecular biochemicals, Mannheim, Germany); and 6 μL H₂O in 20 μL of total volume.

The reactions were incubated 15 minutes at 37° C. The desired reaction product, a blocked oligonucleotide to which a ddUTP-biotin is attached at its 3′ end, is referred to below as G2-ddUTP-biotin or G3-ddUTP-biotin, etc. . . . This is a slight deviation from the manufacturer's recommended protocol in that the concentration of ddUTP-biotin is 10-fold lower than suggested. Surprisingly, it was found that the yield of amplified ligation product was higher under these conditions. This may be because larger amounts of ddUTP-biotin compete with blocked oligonucleotide for biotin-binding sites on the beads.

Three aliquots of 25 μL of Magnabind streptavidin beads (Pierce, Rockford, Ill.) were washed once with 50 μL of 2×B&W buffer (10 mM Tris-HCl pH 7.5, 1 mM EDTA, 2M NaCl) and resuspended in 25μL 2×B&W. Then 20 μL of the G2-ddUTP-biotin reaction were added to 25 μL of washed beads. The same was done for the G3-ddUTP-biotin and G4-ddUTP-biotin reactions. The beads and blocked oligonucleotide mixtures were incubated for 30 minutes at 43° C., mixing on occasion to allow binding of the oligonucleotides to the beads. Supernatants were removed and discarded. 50 μL of 20 μM biotin was added and incubated at 43° C. for 10 minutes to block unoccupied biotin-binding sites on the beads. Beads were washed once with 100 μL of 2×B&W buffer. Beads were washed once in 25 μL of 1×T4 RNA ligase reaction buffer. As a result, the resuspended beads were loaded with desired oligonucleotides (G2, G3 and G4) and ready for ligation.

Ligation & amplification: G1+G2

The following reagents were added to G2 beads: 10 μL of 25 μM oligo G1; 3 μL 200 μM rATP; 1 μL T4 RNA ligase; 3 μL 10× RNA ligase buffer; and 13 μL H₂O for a final total volume of 30 μL. The ligation was allowed to proceed over night at 25° C. Similarly and in parallel, G3 beads were ligated to G2 oligonucleotides (25 μM) and G4 beads were ligated to G3 oligonucleotide (25 μM). Beads were washed twice with 100 μL of 2×B&W and resuspended in 20 μL of H₂O.

To amplify the G1-G2 ligation product, PCR was performed on washed G1+G2 beads by adding: 2.5 μL of bead suspension; 2 μL of 25 μM of oligonucleotide pcrG1; 21 μL of 25 μM G2-; 5 μL 10× buffer (Thermopol buffer supplied with Vent); 5 μL 2 mM dNTPs (each); 1 μL Vent (2000 U/mL, from New England Biolabs, Inc., Beverly, Mass.); and 32.5 μL H₂O for a final total volume of 50 μL.

The cycling conditions for the PCR were: 90 seconds at 95° C. followed by 25 cycles of three successive incubations for 15 seconds at 95° C., 15 seconds at 50° C. and 15 seconds at 72° C., followed by a 120 second incubation at 72° C. The G2-G3 and G3-G4 ligation products were amplified similarly, except that G2-G3 bead suspension was used to provide the template of the G2-G3 amplification and G3-G4 bead suspension was used to provide the template of the G3-G4 amplification. Also, instead of using pcrG1 and G2- as primers, G2 itself and G3- were used to amplify G2-G3. Similarly, oligonucleotides G3 and G4- were used to amplify G3-G4.

Only G1-G2 ligation product was observed after this incubation, as determined by PAGE of 2.5 μL aliquots of the PCRs (4%-20% TBE gradient PAGE supplied by Invitrogen, Carlsbad, Calif.). DNA was visualized using SYBR Green I dye according to the manufacturer's instructions (BioWhittaker, Walkersville, Md.). An additional 0.5 μL of Vent polymerase was added to each PCR tube and the samples were subjected to a “touch-down” temperature cycling protocol: 90 seconds at 95° C. followed by 25 cycles of three successive incubations for 15 seconds at 95° C., 20 seconds at 50 to 40° C. and 20 seconds at 72° C., followed by a 120 second incubation at 72° C. In a touch-down PCR, the annealing temperature is decreased by a fixed amount at each cycle. In this case, the annealing temperature was decreased from 50 to 40° C. over 25 cycles (0.4° C./cycle). Examination of aliquots of the resulting samples by PAGE showed bands of the expected molecular weight (MW) for each of the three amplification samples.

Each PCR product was electrophoresed in an agarose gel, excised and purified using a Qiaquick gel extraction kit (Qiagen, Valencia, Calif.). The resulting DNA samples can conveniently be referred to as G1-G2, G2-G3 and G3-G4.

Amplification by PCR was carried out by mixing: 1 μL of G1-G2; 2 μL of G2-G3; 2 μL of G3-G4; 2 μL of 25 μM of oligo pcrG1; 2 μL of 25 μM G4-; 5 μL 10× buffer; 5 μL 2 mM dNTPs (each); 1 μL Vent; and H₂O for a final total volume of 50 μL.

PCR was performed using the following touch-down conditions: 90 seconds at 95° C. followed by 30 cycles of three successive incubations for 15 seconds at 95° C., 20 seconds at 55 to 50° C. and 30 seconds at 72° C., followed by a 120 second incubation at 72° C.

The desired amplification product, referred to as G1234 ( 320 bp), was observed by electrophoresis of an aliquot of the PCR reaction on an agarose gel. The PCR product was cloned into vector pCR2.1-TOPO (Invitrogen) according to the manufacturer's instructions. The PCR product was also cloned into plasmid pGFP (Clontech, Palo Alto, Calif.) by restriction digestion of Kpn I and Bsr GI sites of both the vector and insert and ligation.

Random TOPO clones of G1234 were sequenced using a model 310 Genetic Analyzer (Applied Biosystems, Foster City, Calif.) with sequencing reagents and instructions provided by the manufacturer. Sequencing revealed that all nine sequences had approximately the desired sequence, except for random point mutations and insertions. For example, in all nine sequences the junction of oligos G1 and G2 had a single insertion of the base adenine (A). Similarly, in some of the nine sequences “A” insertions were also observed at junctions G2-G3 and G3-G4. It will be shown in the next example that these insertions can be greatly reduced if not eliminated.

Clones of pGFP were screened for expression of functional synthetic GFP by assaying colonies for fluorescence. One clone, called SGFP1, was found to be fluorescent and its DNA was sequenced. Compared to wildtype (WT) GFP, this sequence was found to differ at three bases. The first difference was encoded in oligonucleotide G3 to distinguish WT GFP from clones of the synthetic G1234, thus confirming that the synthesis method successfully assembled oligonucleotides in the correct order to yield a functional gene fragment. The other two differences were in codon 87 of the GFP orf (open reading frame). These substitutions, possibly due to errors accumulated during the amplification steps of this protocol, cause the substitution of histidine for alanine at position 87 (A87H). Clone SGFP1 showed a delayed fluorescence phenotype which may be due to this mutation. More specifically, if colonies expressing SGFP1 are assayed for fluorescence after 24 hours of growth on LB plates containing 100 μg/mL of ampicillin, no fluorescence is observed. However, an additional 24 hours of incubation is sufficient for the colonies to become fluorescent.

### Example 2

### Preparation of a Library of Polynucleotides

Ligation of contiguous oligonucleotides using a preferred, multi-step process rather than the ligation method described in Example 1, is described below. In addition, ligation of mixtures of oligonucleotides to generate a combinatorial library of sequences, as in FIG. 2, is illustrated.

In addition to the oligonucleotides described in example 1, new oligonucleotides were synthesized and purified as described above:

R1 (100 mer):

| | |
|---|---|
| R1 (100mer): | |
| AGAGGATCCCCGGGTACCGGTAGAAAAAATGAGGT | (SEQ ID NO:38) |
| CTTCCAAGAATGTTATCAAGGAGTTCATGAGGTTT | |
| AAGGTTCGCATGGAAGGAACGGTCAATGGG | |

R2 (60 mer, 5′ phosphorylated):

| | |
|---|---|
| R2 (60mer, 5′ phosphorylated): | |
| CACGAGTTTGAAATAGAAGGCGAAGGAGAGGGGAG | (SEQ ID NO:39) |
| GCCATACGAAGGCCACAATACCGTA | |
| R3 (63mer, 5′ phosphorylated): | |
| AAGCTTAAGGTAACCAAGGGGGGACCTTTGCCATT | (SEQ ID NO:40) |
| TGCTTGGGATATTTTGTCACCACAATTT | |
| R4 (93mer, 5′ phosphorylated): | |
| CAGTATGGAAGCAAGGTATATGTCAAGCACCCTGC | (SEQ ID NO:41) |
| CGACATACCAGACTATAAAAAGCTGTCATTTCCTG | |
| AAGGATTTGTACAGGAAAGGGTC | |
| R2- (18mer): | |
| TACGGTATTGTGGCCTTC | (SEQ ID NO:42) |
| R3- (21mer): | |
| AAATTGTGGTGACAAAATATC | (SEQ ID NO:43) |
| R4- (20mer): | |
| GACCCTTTCCTGTACAAATC | (SEQ ID NO:44) |

Once assembled in the correct order, 5′-R1-R2-R3-R4-3′, the resulting 316 bp-long polynucleotide encoded almost the entire 5′ half of the red fluorescent protein (referred to herein as RFP, but more generally known as DsRed) gene of a Discosoma (coral) species (Matz, et al., Nat. Biotechnol., 1999, 17, 956). Moreover, a combinatorial library of fluorescent protein sequences was generated to yield several examples of the 16 possible combinations of R1 or G1, R2 or G2, R3 or G3, R4 or G4. For instance, possible sequences include R1-G2-R3-G4, R1-R2-R3-R4, G1-G2-R3-R4, etc. . . .

Loading Beads with Oligos

Oligonucleotides were resuspended in water to yield 25 μM solutions. Then ddUTP-biotin labeling of a mixture of G2 and R2, as well as mixtures of G3 and R3 and G4 and R4 was performed by mixing in 3 separate tubes: 2 μL each of 25 μM oligonucleotide G2 & R2, G3 & R3 or G4 & R4; 4 μL 5× buffer provided with enzyme; 4 μL CoCl₂ 25 mM, 1 μL 100 μM ddUTP-biotin (biotin-ε-aminocaproyl-γ-aminobutyryl-[5-(3aminoallyl)-2′,3′-dideoxyuridine-5′-triphosphate, Roche Molecular biochemicals, Mannheim, Germany); 1 μL terminal transferase (50 U/mL, Roche Molecular biochemicals, Mannheim, Germany); and 6 μL H₂O in 20 μL of total volume. The reactions were incubated 15 minutes at 37° C. The desired reaction product, a mixture of 2 blocked oligonucleotides to which a ddUTP-biotin is attached at their 3′ ends, is referred to below as RG2-ddUTP-biotin or RG3-ddUTP-biotin, etc. . . .

Three aliquots of 25 μL of Magnabind streptavidin beads (Pierce, Rockford, Ill.) were washed once with 50 μL of 2×B&W buffer (10 mM Tris-HCl pH 7.5, 1 mM EDTA, 2M NaCl) and resuspended in 25 μL 2×B&W. Then 20 μL of the RG2-ddUTP-biotin reaction were added to 25 μL of washed beads. The same was done for the RG3-ddUTP-biotin and RG4-ddUTP-biotin reactions. The beads and blocked oligonucleotide mixtures were incubated 30 minutes at 43° C., mixing on occasion to allow binding of the oligonucleotides to the beads.

Supernatants were removed and discarded. 50 μL of 20 μM biotin was added and incubated at 43° C. for 10 minutes to block unoccupied biotin-binding sites on the beads. Beads were washed once with 100 μL of 2×B&W buffer. Beads were washed once in 25 μL of 1×T4 RNA ligase reaction buffer. As a result, the resuspended beads were loaded with desired oligonucleotides (RG2, RG3 and RG4) and ready for ligation.

Multi-Step Ligation & Amplification: RG1+RG2

The following reagents were added to RG2 beads: 2 μL 200 μM rATP; 1 μL T4 RNA ligase; 2 μL 10× RNA ligase buffer; and 15 μL H₂O for a final total volume of 20 μL. This adenylylation was allowed to proceed 6 hours at 25° C. The beads were then washed once in 50 μL of H₂O and resuspended in: 5 μL each of 25 μM G1 and R1; 1 μL T4 RNA ligase; 2 μL 10× RNA ligase buffer; 7 μL H₂O for a final volume of 20 μL. This reaction was incubated over night at 25° C. Similarly and in parallel, RG3 beads were ligated in two steps to a mixture of R2 and G2 oligos (25 μM) and RG4 beads to a mixture of R3 and G3 oligos (25 μM). Beads were washed twice with 100 μL of 2×B&W and resuspended in 20 μL of H₂O.

To amplify the RG1-RG2 ligation products, PCR was performed on washed RG1+RG2 beads by adding: 2.5 μL of bead suspension; 2 μL of 25 μM of oligonucleotide pcrG1; 1 μL of 25 μM R2-; 1 μL of 25 μM G2-; 5 μL 10× buffer (Thermopol buffer supplied with Vent); 5 μL 2mM dNTPs (each); 1 μL Vent (2000 U/mL, from New England Biolabs, Inc., Beverly, Mass.); and H₂O to a final total volume of 50 μL.

The cycling conditions for the touch-down PCR were: 90 seconds at 95° C. followed by 25 cycles of three successive incubations for 15 seconds at 95° C., 20 seconds at 53 to 43° C. and 20 seconds at 72° C., followed by a 120 second incubation at 72° C. The RG2-RG3 and RG3-RG4 ligation products were amplified similarly, except that RG2-RG3 bead suspension was used to provide the template of the RG2-RG3 amplification and RG3-RG4 bead suspension was used to provide the template of the RG3-RG4 amplification. Also, instead of using pcrG1, R2- and G2- as primers, 1 μL each of G2, R2, R3- and G3- (all 25 μM) were used to amplify RG2-RG3. Similarly, oligonucleotides G3, R3, R4- and G4- were used to amplify RG3-RG4. All three PCRs produced a band of the expected size, as determined by PAGE of 2.5 μL aliquots of the PCRs (4%-20% TBE gradient PAGE supplied by Invitrogen, Carlsbad, Calif.). DNA was visualized using SYBR Green I dye according to the manufacturer's instructions (BioWhittaker, Walkersville, Md.).

Each PCR product was electrophoresed in an agarose gel, excised and purified using a Qiaquick gel extraction kit (Qiagen, Valencia, Calif.). The resulting DNA samples can conveniently be referred to as RG1-RG2, RG2-RG3 and RG3-RG4.

Assembly and amplification by PCR was carried out by mixing: 8 μL of RG1-RG2; 4 μL of RG2-RG3; 4 μL of RG3-RG4; 2 μL of 25 μM of oligonucleotide pcrG1; 1 μL of 25 μM G4-; 1 μL of 25 μM R4-; 5 μL 10× buffer; 5 μL 2 mM. dNTPs (each); 1 μL Vent; and H₂O was added to a final total volume of 50 μL. PCR was performed using the following touch-down conditions: 90 seconds at 95° C. followed by 20 cycles of three successive incubations for 15 seconds at 95° C., 20 seconds at 55 to 50° C. and 30 seconds at 72° C., followed by a 120 second incubation at 72° C.

The desired amplification product, referred to as RG1234, ( 320 bp) was observed by electrophoresis of an aliquot of the PCR reaction on an agarose gel. The PCR product was cloned into vector pCR2.1-TOPO (Invitrogen) according to the manufacturer's instructions. The PCR product was also cloned into plasmid pGFP (Clontech, Palo Alto, Calif.) by restriction digestion of Kpn I and Bsr GI sites of both the vector and insert and ligation.

Random TOPO clones of RG1234 were sequenced using a model 310 Genetic Analyzer (Applied Biosystems, Foster City, Calif.) with sequencing reagents and instructions provided by the manufacturer. Sequencing of eight different clones revealed that they were the product of a stochastic assembly of oligonucleotides (see Table 1). Various combinations of building blocks were clearly observed. Most sequences carried some defects such as deletions or insertions. One sequence, RG9, showed only a few point mutations, providing an example of a sequence in which all junctions were perfect. Moreover, in contrast with the previous example, only one of the 24 junctions described in Table 1 (8 sequences×3 junctions each) had an unwanted ‘A’ insertion, showing the benefit of a multi-step ligation.

TABLE 1
Sequencing results of random TOPO clones (RG1 to RG9) and functional pGFP
clone (RG100) of RG1234.

| | Oligonucleotide | Oligonucleotide | Oligonucleotide | Oligonucleotide |
|---|---|---|---|---|
| | at sequence | at sequence | at sequence | at sequence |
| Clone | position 1 | position 2 | position 3 | position 4 |
| name | (comments) | (comments) | (comments) | (comments) |
| RG1 | R1 (truncated 3′ | R2 (no defects) | R3 (no defects) | R4 (1 point |
| | end) | | | mutation) |
| RG2 | G1 (truncated 3′ | R2 (no defects) | R3 (A insert at 3′ | G4 (2 point |
| | end) | | end) | mutations) |
| RG3 | R1 (2 point | R2 (no defects) | R3 (3 nt deletion | R4 (4 point |
| | mutations) | | at 5′ end and 7 nt | mutations) |
| | | | deletion at 3′ end) | |
| RG4 | R1 (1 point | R2 (no defects) | R3 (2 point | R4 (no defects) |
| | mutation) | | mutations, 2 | |
| | | | insertions, 1 C | |
| | | | insertion at 3′ | |
| | | | end) | |
| RG5 | G1 (1 deletion, 1 | R2 (no defects) | R3 (no defects) | R4 (1 point |
| | point mutation) | | | mutation) |
| RG7 | R1 (trucated at 3′ | R2 (last nt | G3 (no defects) | R4 (1 insertion, 1 |
| | end) | deleted) | | point mutation) |
| RG8 | R1 (4 mutations, | R2 (no defects) | R3 (no defects) | G4 (1 point |
| | 10 nt deleted at 3′ | | | mutation) |
| | end) | | | |
| RG9 | R1 (4 mutations) | R2 (no defects) | R3 (no defects) | G4 (6 point |
| | | | | mutations) |
| RG100 | G1 (no defects) | G2 (no defects) | G3 (no defects) | G4 (no defects) |
| (pGFP | | | | |
| clone) | | | | |

Clones of pGFP were screened for expression of functional synthetic GFP or functional GFP/RFP recombinants by assaying colonies for fluorescence under illumination at different wavelengths. Several clones showing fluorescence typical of WT GFP were identified readily. The DNA of one fluorescent clone, called RG100, was sequenced. Compared to wildtype (WT) GFP, this sequence was found to differ at one base. The difference was the mutation encoded in oligo G3 to distinguish WT GFP from clones resulting from the assembly process. RG100 therefore provides an example of, not only a functional sequence, but exactly the desired sequence resulting from the correct assembly of a combinatorial mixture of oligos. Also, the nine sequences described in this example illustrate that all 8 possible oligonucleotides were found in products of the assembly process.

### Example 3

### Oligonucleotide Design for the Preparation of Libraries from a Set of Phylogenetically Related Polynucleotides

Subtilisin Carlsberg, a member of the subtilase family of enzymes, from the organism Bacillus licheniformis is found to cleave an ester X. The goal is to improve the weak activity of this subtilisin towards substrate X.

The amino acid sequence of the enzyme (accession number 995625) was used to identify related sequences from the public database of sequences available online by performing a BLAST search (www.ncbi.nlm.nih.gov/BLAST/). Twenty-five sequences were chosen manually from 100 sequences obtained in the BLAST search results. In an alternative embodiment, the selection process may be automated.

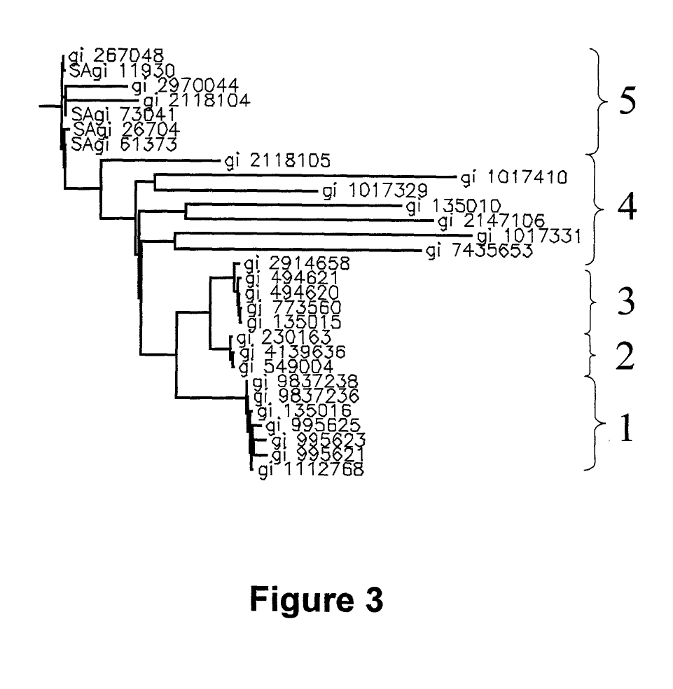

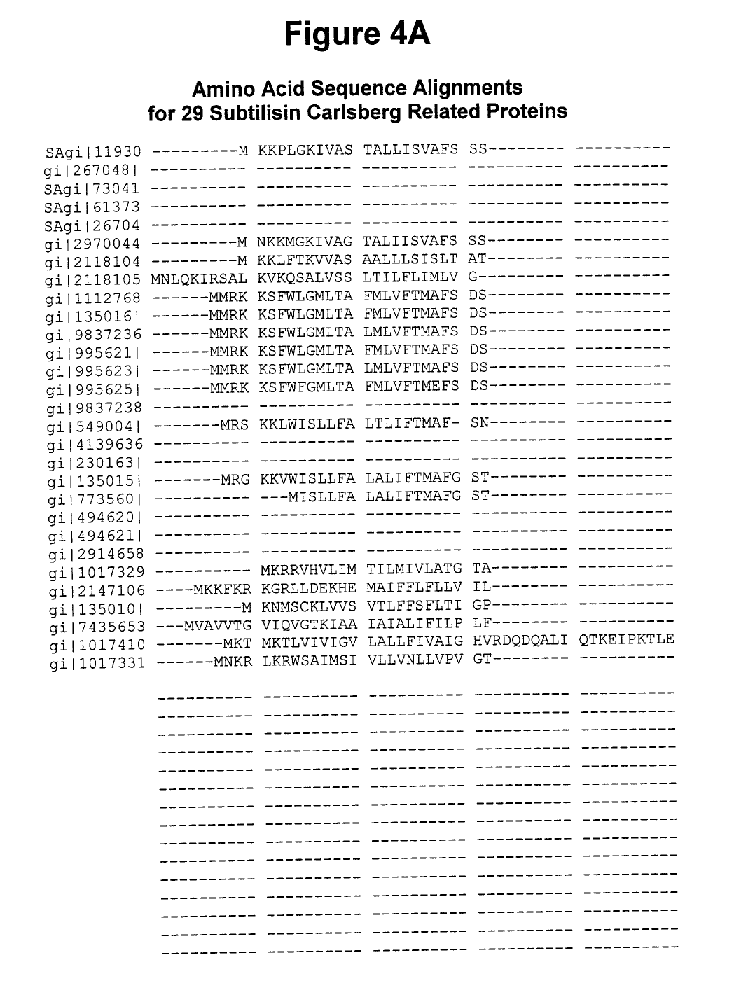

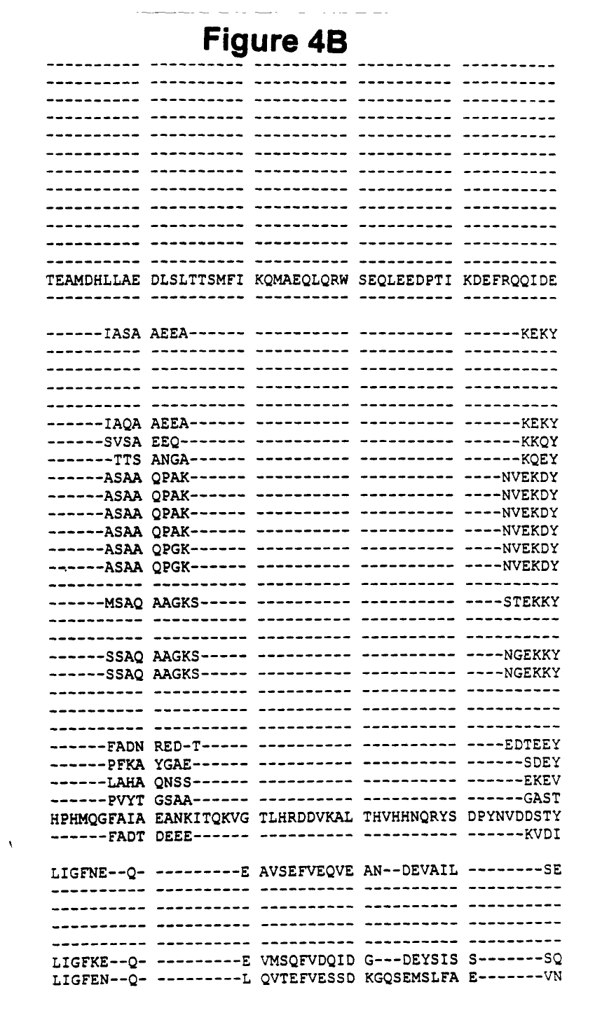

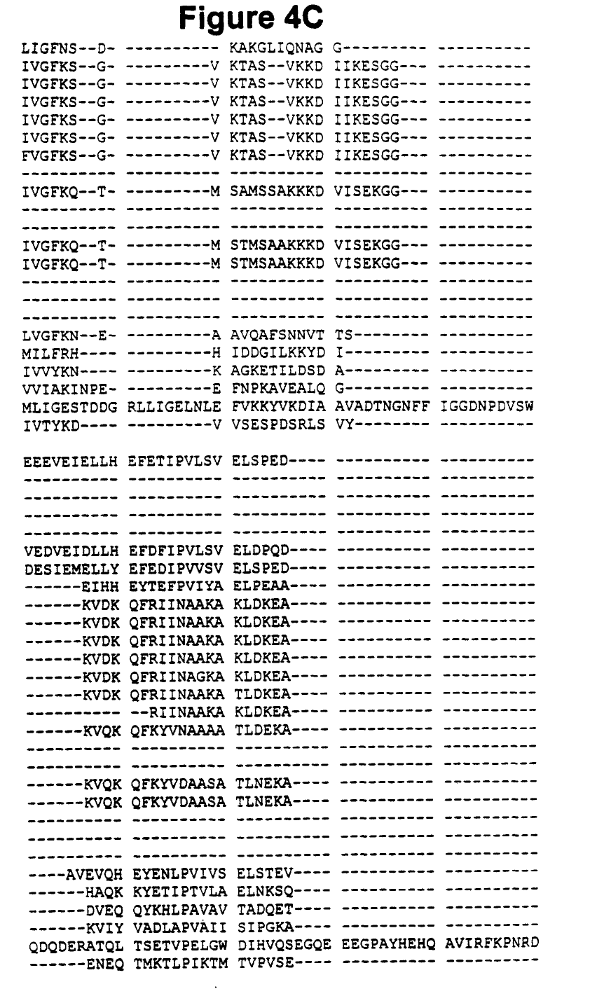

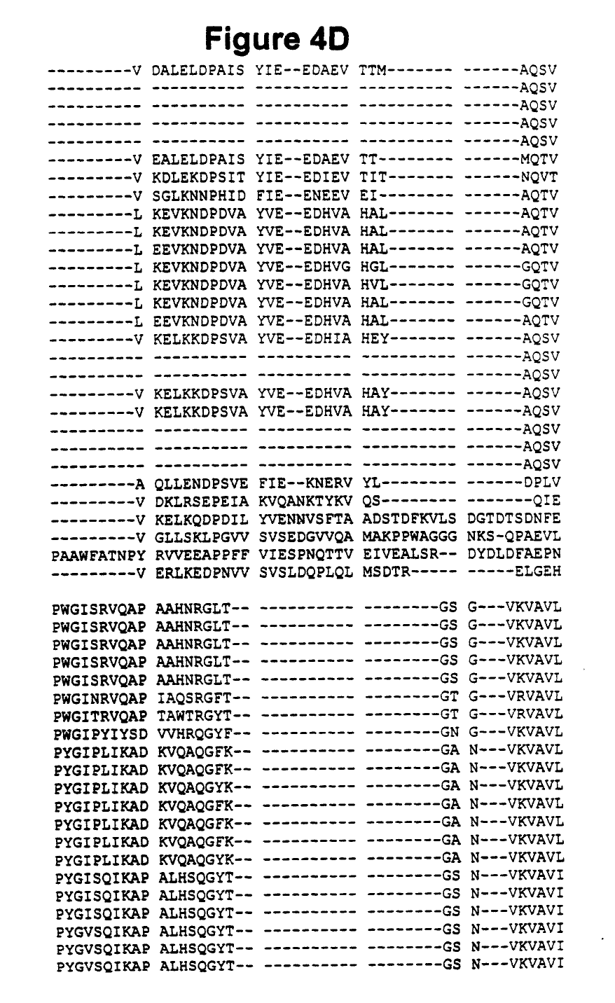

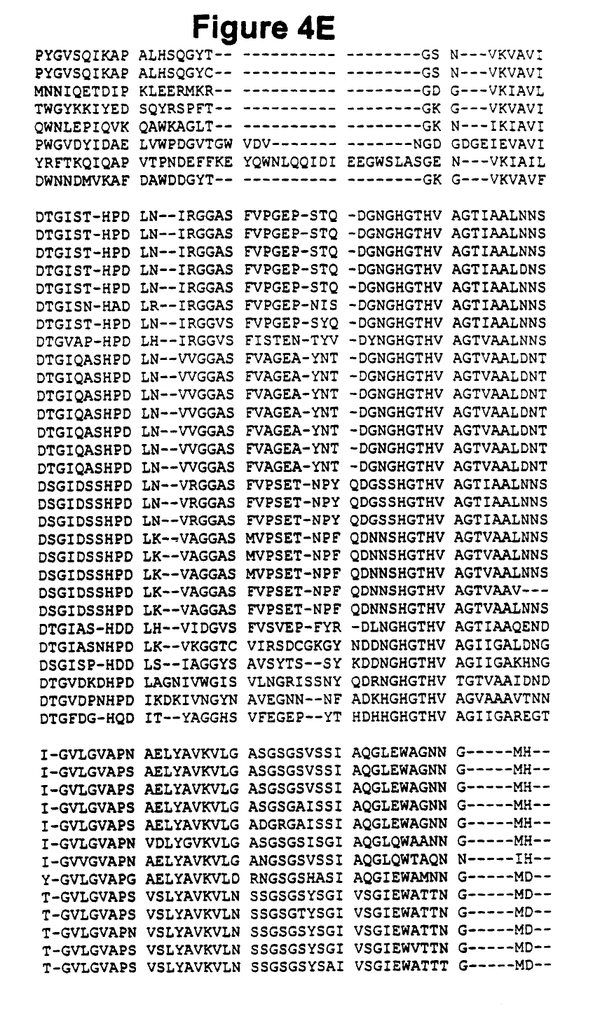

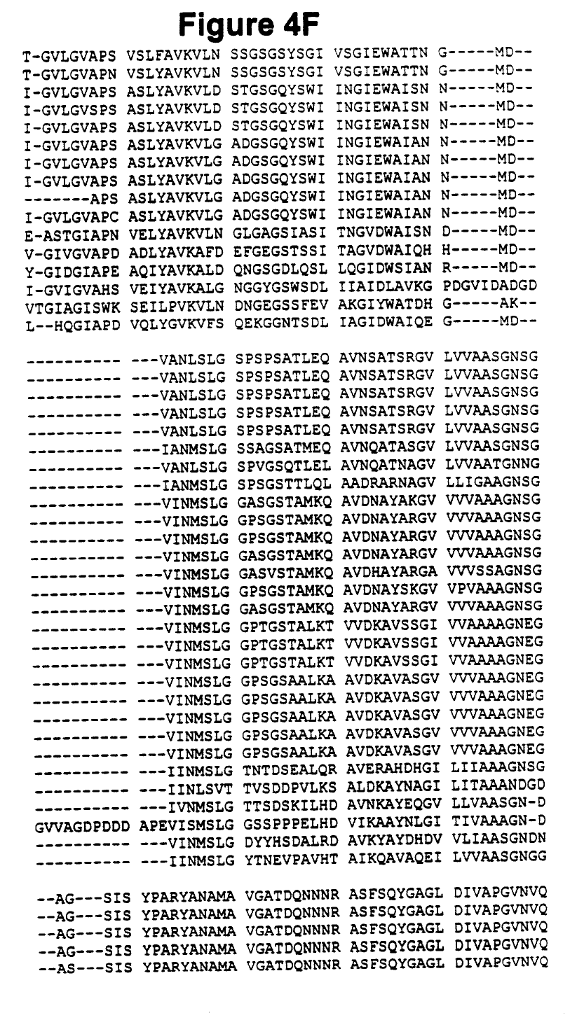

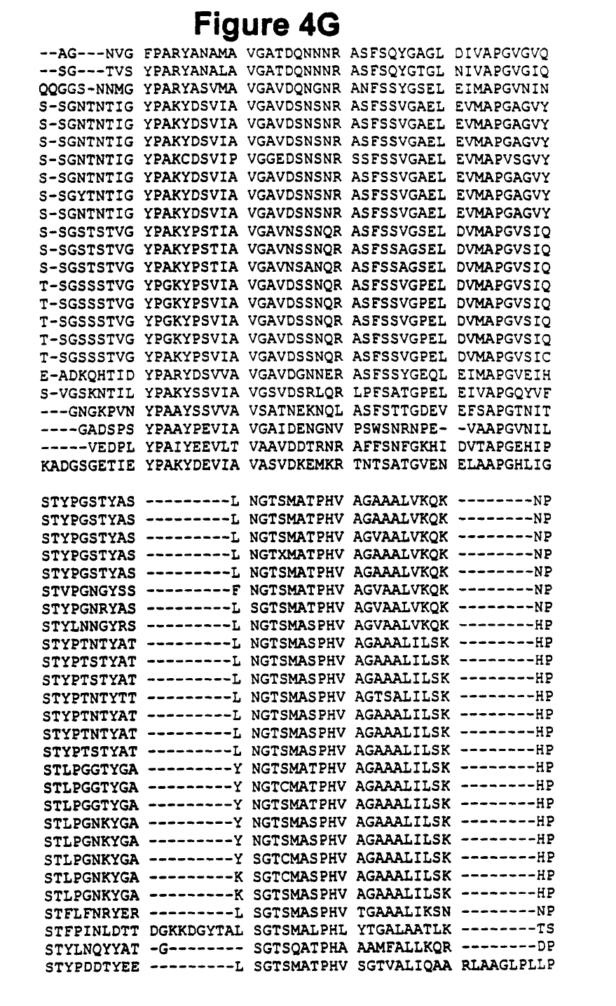

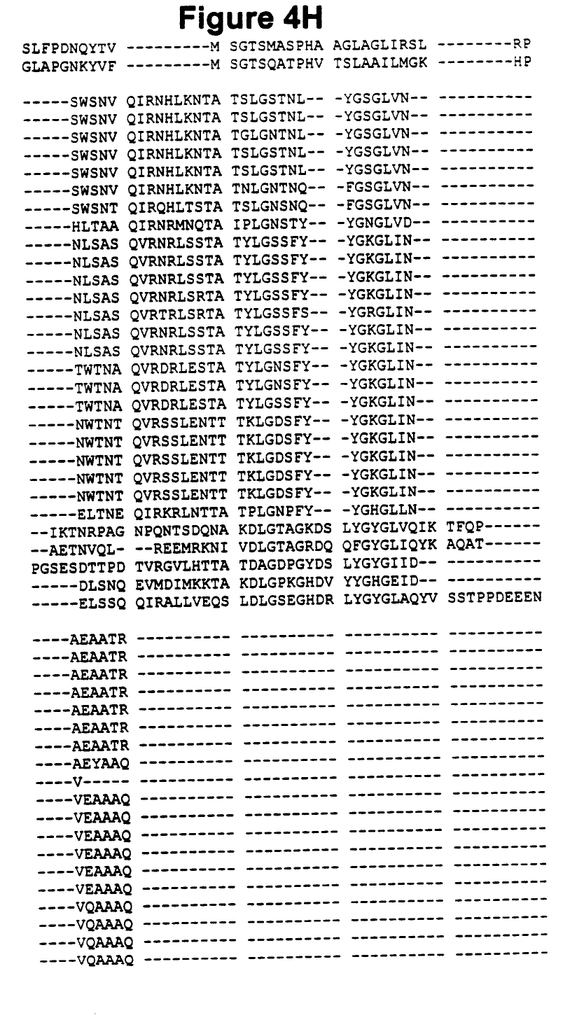

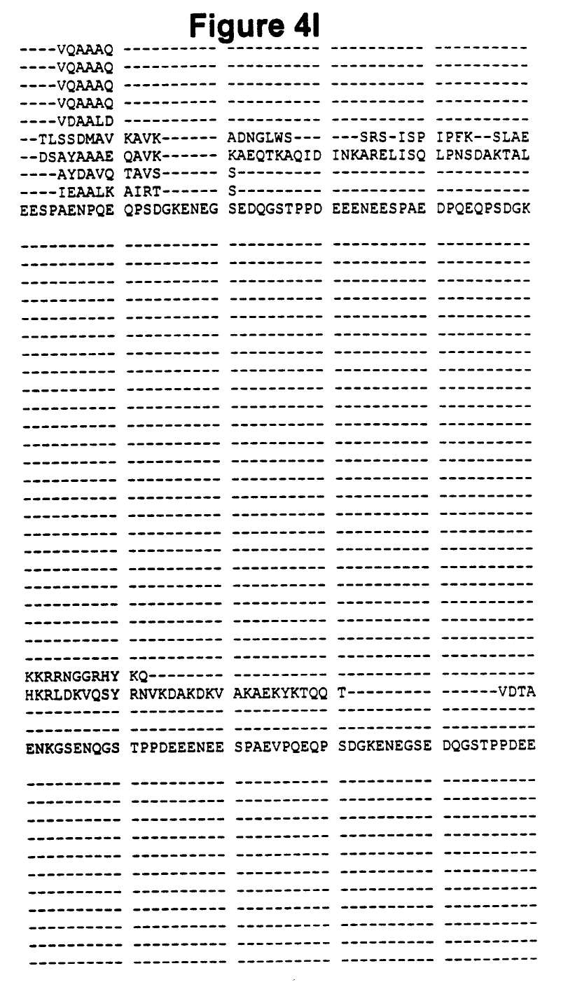

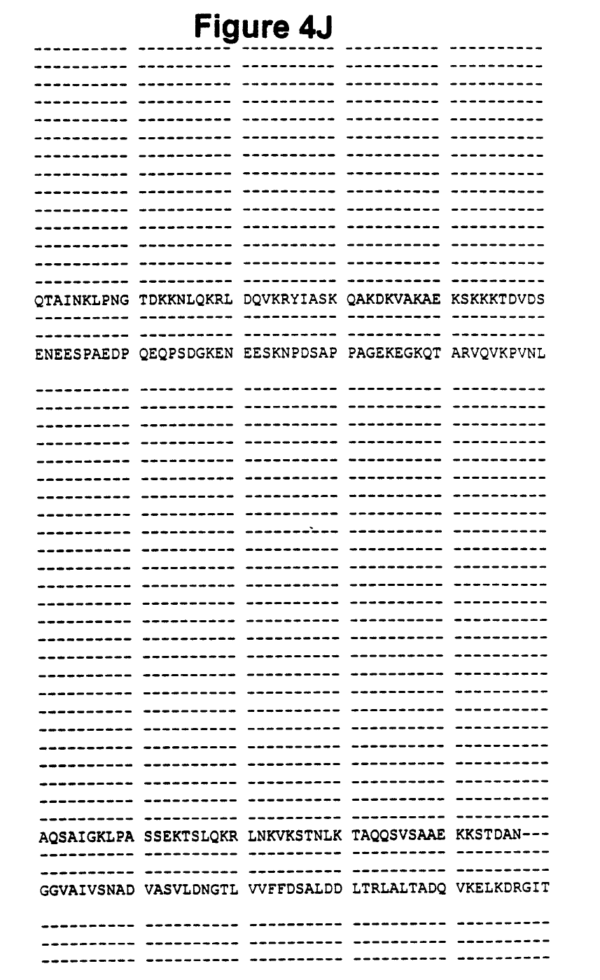

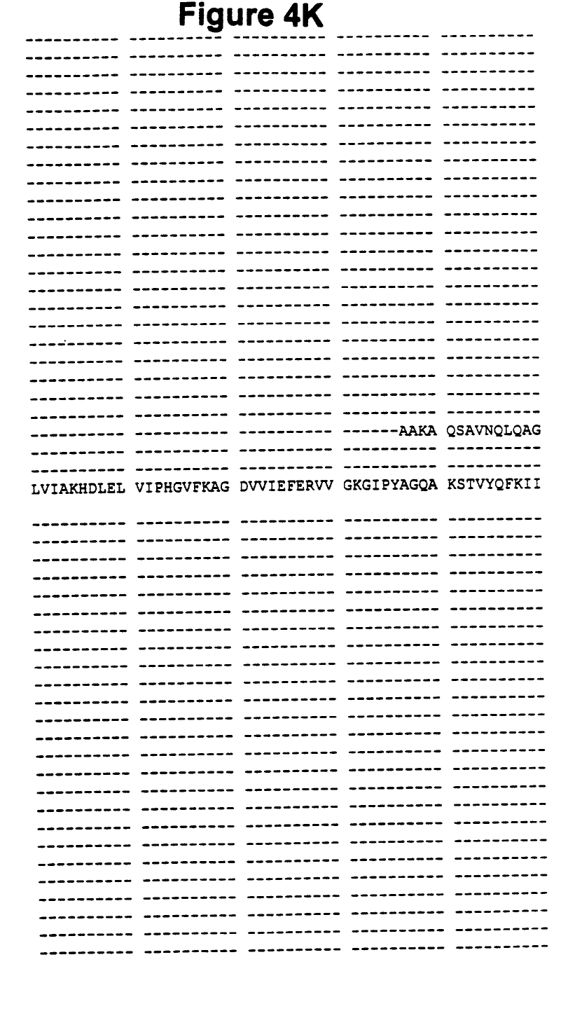

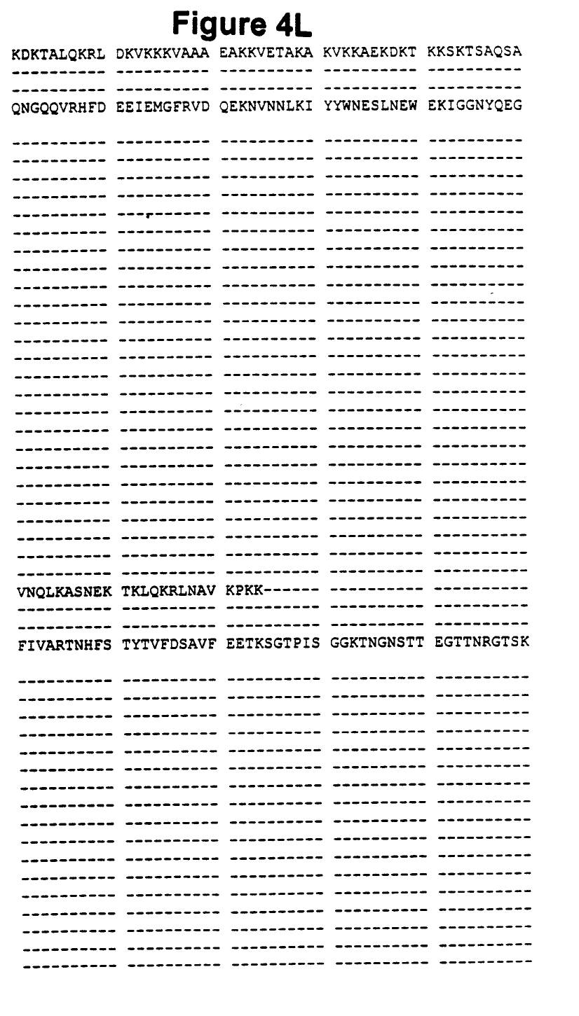

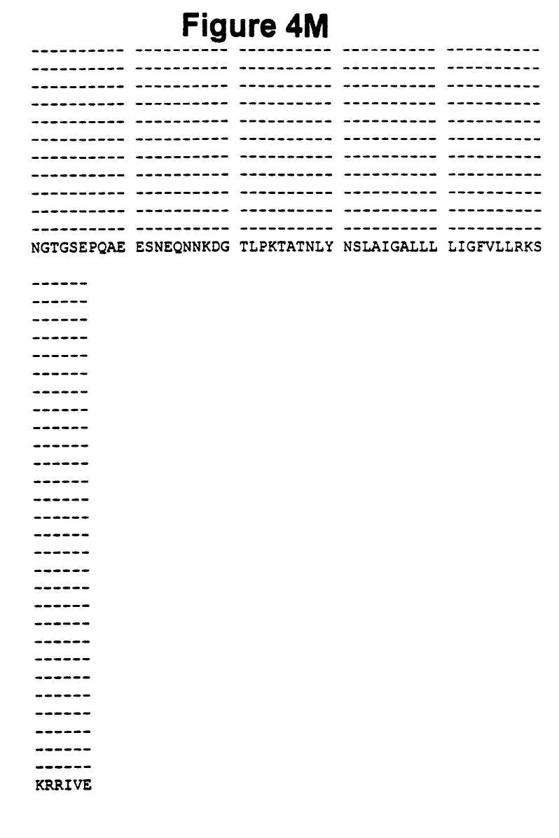

The 25 sequences were analyzed using ClustalW and the Phylip software package and it was found that the 25 sequences can be broken down into 5 families. One of these families (Savinase-related, accession number 119308) is only represented by a single member so an additional four sequences are added to the 25. A further analysis is performed using ClustalW v. 1.7 and the Phylip software package. The resulting phenogram is depicted in FIG. 3, showing the five family groups: family 1 corresponding to sequences related to Alcalase (subtilisin Carlsberg from Bacillus lichenifomis), family 2 corresponding to sequences related to chain A of the mesentericopeptidase (E.C.3.4.21.14) peptidyl peptide hydrolase complex (gi230163), family 3 corresponding to subtilisin BPN (subtilisin Novo; gi135015), family 4 corresponding to sequences distantly related to families 1 to 3, and family 5 corresponding to Savinase (gi267048) and related sequences such as the subtilisin of Bacillus lentus. FIGS. 4A-4M show ClustalW alignment of all 29 sequences where dashes indicate gaps in the alignment.

The sequence of subtilisin Carlsberg (family 1; gi1112768) is divided arbitrarily into 19 sequence fragments of 20 amino acids in length, or 60 bp at the nucleotide level. In an alternative embodiment, a more sophisticated approach could be taken to break down the sequence into fragments. For example, the 19 fragments could be modified so that their 5′ ends correspond to pyrimidines (which are preferred by T4 RNA ligase) but not purines. This would mean that fragments would generally be of slightly different lengths.

The sequences of family 1 were aligned together. Within families, differences between the sequences generally limit themselves to point mutations. Such slight differences can readily be encompassed by the assembly of degenerate oligonucleotides wherein two or more bases are simultaneously encoded by the DNA synthesizer used to make the oligonucleotide. In cases where more than simple single-nucleotide differences must be encoded by a degenerate oligonucleotide, the program CyberDope, available online at www.kairos-scientific.comlsearchable/cyberdope.html, can be used to design appropriate degenerate codons. These degenerate codons are selected by the program to encode complex combinations of amino acids. Oligonucleotides, including those that are degenerate, can be are commercially available from companies such as Operon Inc. (Alameda, Calif.). Therefore, the 7 sequences of family 1 were described by 19 oligonucleotides, most of which have 2 degenerate nucleotide positions. Oligonucleotide number 13 was the most complex, encoding 7 mutations requiring 8 nucleotide degeneracies. Although the combinatorial complexity of the 19 degenerate oligonucleotides exceeds 2⁴², or more than 4×10¹² possible sequences, the amino acid substitutions are quite conservative, such that most combinations will likely yield functional subtilisins with a variety of phenotypes.

Similarly, the sequences of family 3 are aligned. As with family 1, the sequences of family 3 differed generally by no more than 2 amino acids in each 20 amino acid sequence fragment. In fact, family 3 showed fewer mutations than family 1. The 5 sequences of family 3 can almost be described by 19 oligonucleotides, most of which have no degeneracies or only one. There are, however, three exceptions due to gaps in the alignment: oligonucleotides 1, 9 and 10. The first oligonucleotide (5′-most) cannot encode both sequences #135015 and #773560, as one is slightly shorter than the other at the 5′ end. Thus, two oligonucleotides (3F1a and 3F1b) were needed for this sequence position. Similarly, a gap in sequence #494621 must be encoded by specifying 4 different oligos: 3F9a, 3F9b, 3F10a and 3F10b. The apparent absence of sequence data at the 5′ end of sequences #2914658, 494620 and 494621 was due to the cleavage of a signal and prosequence. Thus, these were assumed to be identical to the sequences of#135015 and 773560.

Using the amino acid sequences encoded by the above polynuclotides of families 1 and 3, degenerate oligonucleotides were designed with the program CyberDope. The resulting oligonucleotides needed to synthesize the orf of families 1 and 3 are listed below. Oligonucleotides are numbered in the order in which they are to be assembled, from the 5′ to the 3′ end. Degeneracies are encoded according to the IUPAC code (described on p.234 of the 2000-2001 New England Biolabs catalog or avilable, for example, online at www.neb.com/neb/tech/tech_resource/miscellaneous/genetic_code.html.

| | |
|---|---|
| 1F1 | |
| ATGATGAGGAAAAAGAGTYTTTGGTTTGGGATGTT | (SEQ ID NO:45) |
| GACGGCCYTTATGCTCGTGTTCACG | |
| 1F2 | |
| ATGGMGTTCAGCGATTCCGCTTCTGCTGCTCAACC | (SEQ ID NO:46) |
| GGSGAAAAATGTTGAAAAGGATTAT | |
| 1F3 | |
| WTTGTCGGATTTAAGTCAGGAGTGAAAACCGCATC | (SEQ ID NO:47) |
| TGTCAAAAAGGACATCATCAAAGAG | |
| 1F4 | |
| AGCGGCGGAAAAGTGGACAAGCAGTTTAGAATCAT | (SEQ ID NO:48) |
| CAACGCGGSAAAAGCGACGCTAGAC | |
| 1F5 | |
| AAAGAAGCGCTTRAGGAAGTCAAAAATGATCCGGA | (SEQ ID NO:49) |
| TGTCGCTTATGTGGAAGAGGATCAT | |
| 1F6 | |
| GTGGSACATGBGTTGGSACAAACCGTTCCTTACGG | (SEQ ID NO:50) |
| CATTCCTCTCATTAAAGCGGACAAA | |
| 1F7 | |
| GTGCAGGCTCAAGGCTWTAAGGGAGCGAATGTAAA | (SEQ ID NO:51) |
| AGTAGCCGTCCTGGATACAGGAATC | |
| 1F8 | |
| CAAGCTTCTCATCCGGACTTGAACGTAGTCGGCGG | (SEQ ID NO:52) |
| AGCAAGCTTTGTGGCTGGCGAAGCT | |
| 1F9 | |
| TATAACACCGACGGCAACGGACACGGCACACATGT | (SEQ ID NO:53) |
| TGCCGGTACAGTAGCTGCGCTTGAC | |
| 1F10 | |
| AATACAACGGGTGTATTAGGCGTTGCGCCAARTGT | (SEQ ID NO:54) |
| ATCCTTGAWTGCGGTTAAAGTACTG | |
| 1F11 | |
| AATTCAAGCGGAAGCGGAASTTACAGCGSAATTGT | (SEQ ID NO:55) |
| AAGCGGAATCGAGTGGGYGACAACA | |
| 1F12 | |
| AMTGGCATGGATGTTATCAATATGAGCCTTGGGGG | (SEQ ID NO:56) |
| ASCATCAGKGTCGACAGCGATGAAA | |
| 1F13 | |
| CAGGCAGTCGACMATGCATATKCTARGGGGGYTGT | (SEQ ID NO:57) |
| CSYTGTAKCTKCTGCAGGGAACAGC | |
| 1F14 | |
| GGATCTTCAGGAWATACGAATACAATTGGCTATCC | (SEQ ID NO:58) |
| TGCGAAATRTGATTCTGTCATCSCG | |
| 1F15 | |
| GTTGGTGSGGWGGACTCTAACAGCAACAGAKCGTC | (SEQ ID NO:59) |
| ATTTTCCAGTGTGGGAGCAGAGCTT | |
| 1F16 | |
| GAAGTCATGGCTCCTGKGKCGGGCGTATACAGCAC | (SEQ ID NO:60) |
| TTACCCAACGARTACTTATRCGACA | |
| 1F17 | |
| TTGAACGGAACGTCAATGGCTTCTCCTCATGTAGC | (SEQ ID NO:61) |
| GGGARCGKCGGCTTTGATCTTGTCA | |
| 1F18 | |
| AAACATCCGAACCTTTCAGCTTCACAAGTCCGCAM | (SEQ ID NO:62) |
| TCGTCTCTCCAGKACGGCGACTTAT | |
| 1F19 | |
| TTGGGAAGCTCCTTCTMTTATGGGARGGGTCTGAT | (SEQ ID NO:63) |
| CAATGTCGAAGCTGCCGCTCAATAA | |

Following is a list of oligonucleotides necessary to assemble a library of family 3-related sequences.

| | |
|---|---|
| 3F1a | |
| ATGAGAGGCAAAAAAGTATGGATCAGTTTGCTGTT | (SEQ ID NO:64) |
| TGCTTTAGCGTTAATCTTTACG | |
| 3F1b | |
| ATGATCAGTTTGCTGTTTGCTTTAGCGTTAATCTT | (SEQ ID NO:65) |
| TACG | |
| 3F2 | |
| ATGGCGTTCGGCAGCACATCCTCTGCCCAGGCGGC | (SEQ ID NO:66) |
| AGGGAAATCAAACGGGGAAAAGAAATAT | |
| 3F3 | |
| ATTGTCGGGTTTAAACAGACAATGAGCACGATGAG | (SEQ ID NO:67) |
| CGCCGCTAAGAAGAAAGATGTCATTTCTGAA | |
| 3F4 | |
| AAAGGCGGGAAAGTGCAAAAGCAATTCAAATATGT | (SEQ ID NO:68) |
| AGACGCAGCTTCAGCTACATTAAAC | |
| 3F5 | |
| GAAAAAGCTGTAAAAGAATTGAAAAAAGACCCGAG | (SEQ ID NO:69) |
| CGTCGCTTTACGTTGAAGAAGAT | |
| 3F6 | |
| CACGTAGCACATGCGTACGCGCAGTCCGTGCCTTA | (SEQ ID NO:70) |
| CGGCGTATCACAAATTAAAGCCCCTGCT | |
| 3F7 | |
| CTGCACTCTCAAGGCTACWSTGGATCAAATGTTAA | (SEQ ID NO:71) |
| AGTAGCGGTTATCGACAGCGGTATC | |
| 3F8 | |
| GATTCTTCTCATCCTGATTTAAAGGTAGCAGGCGG | (SEQ ID NO:72) |
| AGCCAGCWTKGTTCCTTCTGAAACA | |
| 3F9a | |
| AATCCTTTCCAAGACAACAACTCTCACGGAACTCA | (SEQ ID NO:73) |
| CGTTGCCGGCACAGTTGCGGCTCTTAAT | |
| 3F9b | |
| AATCCTTTCCAAGACAACAACTCTCACGGAACTCA | (SEQ ID NO:74) |
| CGTTGCCGGCACAGTTGCGGCTGTT | |
| 3F10a | |
| AACTCAATCGGTGTATTAGGCGTTGCGCCAWGTGC | (SEQ ID NO:75) |
| ATCACTTTACGCTGTAAAAGTTCTC | |
| 3F10b | |
| GCGCCATCAGCATCACTTTACGCTGTAAAAGTTCTC | (SEQ ID NO:76) |
| 3F11 | |
| GGTGCTGACGGTTCCGGCCAATACAGCTGGATCAT | (SEQ ID NO:77) |
| TAACGGAATCGAGTGGGCGATCGCA | |
| 3F12 | |
| AACAATATGGACGTTATTAACATGAGCCTCGGCGG | (SEQ ID NO:78) |
| ACCTTCTGGTTCTGCTGCTTTAAAA | |
| 3F13 | |
| GCGGCAGTTGATAAAGCCGTTGCATCCGGCGTCGT | (SEQ ID NO:79) |
| AGTCGTTGCGGCAGCCGGTAACGAA | |
| 3F14 | |
| GGCACTTCCGGCAGCTCAAGCACAGTGGGCTACCC | (SEQ ID NO:80) |
| TGSGAAATACCCTTCTGTCATTGCA | |
| 3F15 | |
| GTAGGCGCTGTTGACAGCAGCAACCAAAGAGCATC | (SEQ ID NO:81) |
| TTTCTCAAGCGTAGGACCTGAGCTT | |
| 3F16 | |
| GATGTCATGGCACCTGGCGTATCTATCYRKAGCAC | (SEQ ID NO:82) |
| GCTTCCTGGAAACAAATACGGGGCG | |
| 3F17 | |
| WAKARTGGTACGTCAATGGCATCTCCGCACGTTGC | (SEQ ID NO:83) |
| CGGAGCGGCTGCTTTGATTCTTTCT | |
| 3F18 | |
| AAGCACCCGAACTGGACAAACACTCAAGTCCGCAG | (SEQ ID NO:84) |
| CAGTTTAGAAAACACCACTACAAAA | |
| 3F19 | |
| CTTGGTGATTCTTTCTACTATGGAAAAGGGCTGAT | (SEQ ID NO:85) |
| CAACGTACAGGCGGCAGCTCAGTAA | |

### Example 4

### Preparation of Libraries, Screening of Library Members, and Recursive Screening

At least three types of libraries of DNA molecules can be constructed based on the oligonucleotides designed in Example 3. One type of library encompasses the sequences of family 1, a second type of library describes family 3, and a third type of library can be constructed which combines the sequences of families 1 and 3. This latter library can be constructed by mixing in equal proportions the oligonucleotides designed for the synthesis of the first and second libraries. In order for this approach to work, the oligonucleotides designed from family 3 should be broken down at homologous sequence positions to the oligonucleotides from family 1. In consideration of the preparation of libraries incorporating both families 1 and 3, it is apparent that a single oligonucleotide requires a larger number of degenerate positions to encode sequences from both families, and that the combinatorial complexity thus generated may disrupt sequence motifs. This disruption of motifs may decrease unacceptably the proportion of functional sequences in the resulting library. Moreover, such large numbers of degenerate positions in an oligonucleotide may interfere with the assembly process, (i.e., during amplification). Thus, the practical issues of complexity must be weighed against the desire for diversity in the preparation of libraries.

Oligonucleotides representing families 2, and 5 are prepared in addition to the oligonucleotides of families 1 and 3 obtained in Example 3, and together are used to assemble a library according to the methods of the present invention, such as in Example 2. Members of family 4 are not included for simplicity. Care is taken to maintain degeneracies throughout the process (i.e., during amplification). The library encompasses four of the five families described in Example 3 (1, 2, 3, & 5) by mixing in equal proportions of oligonucleotides, one part from each family, at each position in the sequence. The assembly results in a combinatorial library encompassing families 1, 2, 3 & 5 by mixing IF1, 2F1, 3F1a, 3F1b, SF1 and linking this mixture to a mixture of 1F2, 2F2, 3F2, SF2, and so on. Roughly, not including degeneracies, the resulting library would encode over 4¹⁹, or 2.7×10¹¹ different possible sequences. Including degeneracies would increase the number well beyond 10¹² sequences.

Using standard methods, the resulting polynucleotide libraries are cloned into an expression vector and expressed in E. coli or Bacillus subtilis. High throughput screening for subtilase activity is carried out according to Ness et al., Nature Biotechnology, 1999, 17, 893, which is herein incorporated by reference in its entirety. A fluorescent derivative analogous to quenched BODIPY dye-labeled casein is prepared with the compound of interest (ester X in this case) (Jones, et al., Anal. Biochem., 1997, 251, 144, which is herein incorporated by reference in its entirety) and used as a substrate to identify new subtilase variants with improved activity towards this substrate. Regardless of the methods used, only a small proportion of the vast number of possible sequences can be screened for activity. Consequently, it is quite possible that the optimal sequence (i.e., the enzyme best able to use compound X as a substrate) will not be rapidly found. Instead, a small population of enzyme variants with some improved ability to catalyze the reaction of interest will be identified.

The individuals of this small population are characterized by DNA sequencing of the gene encoding the improved variant according to standard methods. It is then determined that this population is predominantly composed of, in a first group of positions along the sequence, oligonucleotides from family 1. Also, it is found that, at a second group of positions along the sequence, the population is composed mostly of oligonucleotides from family 3. Using this information, a new combinatorial library of polynucleotides is synthesized wherein the first group of positions are synthesized exclusively using oligos from family 1 and the second group of positions exclusively using oligonucleotides from family 3. The resulting combinatorial mixture of polynucleotides is cloned and expressed as before and assayed as before. New variants with superior properties (in this case, greater activity towards substrate X) to the original population of variants are found. If necessary, these variants may be used to design a further combinatorial population of variants.

What is claimed is:

1. A method of preparing a polynucleotide having a target sequence from a plurality of oligonucleotides, said method comprising:
(a) coupling said oligonucleotides to form a plurality of coupled oligonucleotides, wherein each of said coupled oligonucleotides represents a region of said polynucleotide and shares at least one terminal region of sequence with at least one other coupled oligonucleotide; and
(b) assembling said polynucleotide by extension of said coupled oligonucleotides.

2. The method of claim 1 wherein said coupling comprises ligating said oligonucleotides with ligase.

3. The method of claim 2 wherein said ligase is T4 RNA ligase.

4. The method of claim 1 wherein at least one of said oligonucleotides of said coupled oligonucleotides is attached to solid support prior to coupling.

5. The method of claim 1 wherein said coupled oligonucleotides are attached to solid support.

6. The method of claim 1 wherein each of said coupled oligonucleotides is amplified prior to assembling said polynucleotide.

7. The method of claim 1 wherein at least one of said oligonucleotides of said coupled oligonucleotides is blocked at one end prior to said coupling.

8. The method of claim 1 wherein said coupled oligonucleotides comprise pairs of oligonucleotides.

9. The method of claim 1 wherein said extension is carried out using overlap PCR.

10. A library of polynucleotides prepared by the method of claim 1.

11. A method of preparing a polynucleotide having a target sequence from a plurality of oligonucleotides, said method comprising:
(a) blocking the 3′ end of each of said oligonucleotides, except for the oligonucleotide comprising the 5′ terminus of said polynucleotide, with a blocking group to form a plurality of blocked oligonucleotides;
(b) coupling the 5′ end of each of said blocked oligonucleotides with the 3′ end of a further oligonucleotide of said plurality of oligonucleotides to form a plurality of coupled oligonucleotides, wherein said further oligonucleotide comprises a portion of said polynucleotide immediately 5′ to the sequence of said blocked oligonucleotide, wherein each of said coupled oligonucleotides shares at least one oligonucleotide with another coupled oligonucleotide; and
(c) assembling said polynucleotide by extension of said coupled oligonucleotides.

12. The method of claim 11 wherein said polynucleotide is DNA, RNA, or DNA/RNA hybrid.

13. The method of claim 11 wherein said oligonucleotides comprise from about 10 to about 200 nucleotides.

14. The method of claim 11 wherein said blocking group comprises solid support.

15. The method of claim 14 wherein said solid support is selected from the group consisting of agarose, polyacrylamide, magnetic beads, polystyrene, polyacrylate, controlled-pore glass, hydroxyethylmethacrylate, polyamide, polyethylene, polyethyleneoxy, and polyethyleneoxy/polystyrene copolymer.

16. The method of claim 11 wherein said blocking group is ddUTP-biotin.

17. The method of claim 11 wherein said coupling is carried out using ligase.

18. The method of claim 17 wherein said ligase is T4 RNA ligase.

19. The method of claim 17 wherein said coupling comprises the steps of contacting said blocked oligonucleotide with ligase and cosubstrate to form activated oligonucleotide, washing said activated oligonucleotide to form washed oligonucleotide, and contacting said washed oligonucleotide with said further oligonucleotide and ligase.

20. The method of claim 11 wherein said coupled oligonucleotides are amplified prior to assembling said polynucleotide.

21. The method of claim 11 wherein said extension is carried out using overlap PCR.

22. A library of polynucleotides prepared by the method of claim 11.

23. A method of coupling a first oligonucleotide with a further oligonucleotide, wherein said first oligonucleotide is attached to solid support, comprising contacting said first oligonucleotide with ligase and cosubstrate to form activated oligonucleotide, washing said activated oligonucleotide to form washed oligonucleotide, and contacting said washed oligonucleotide with said further oligonucleotide and ligase.

24. The method of claim 23 wherein said ligase is T4 RNA ligase.

25. The method of claim 23 wherein said cosubstrate is ATP.

26. A method of preparing a library of polynucleotides having a plurality of target sequences from a plurality of oligonucleotides, wherein each of said polynucleotides shares a plurality of predetermined sequence positions occupied by said oligonucleotides, and wherein each of said polynucleotides comprises a different oligonucleotide in at least one predetermined sequence position, said method comprising:
(a) coupling said oligonucleotides to form a plurality of coupled oligonucleotides, wherein each of said coupled oligonucleotides represents a region of at least one of said polynucleotides and shares at least one terminal region of sequence with at least one other coupled oligonucleotide; and
(b) assembling said library of polynucleotides by extension of said coupled oligonucleotides.

27. The method of claim 26 wherein said coupling comprises ligating said oligonucleotides with ligase.

28. The method of claim 27 wherein said ligase is T4 RNA ligase.

29. The method of claim 26 wherein at least one of said oligonucleotides of said coupled oligonucleotides is attached to solid support prior to coupling.

30. The method of claim 26 wherein said coupled oligonucleotides are attached to solid support.

31. The method of claim 26 wherein each of said coupled oligonucleotides is amplified prior to assembling said polynucleotide.

32. The method of claim 26 wherein at least one of said oligonucleotides of said coupled oligonucleotides is blocked at one end prior to said coupling.

33. The method of claim 26 wherein said coupled oligonucleotides comprise pairs of oligonucleotides.

34. The method of claim 26 wherein said extension is carried out using overlap PCR.

35. The method of claim 26 wherein said plurality of oligonucleotides is derived from a parent set of polynucleotides having at least one common property.

36. The method of claim 35 wherein said common property is sequence homology.

37. The method of claim 35 wherein said common property is enzyme activity.

38. The method of claim 35 wherein said common property is ligand binding.

39. The method of claim 35 wherein said set of polynucleotides is optimized.

40. A method of preparing a library of polynucleotides having a plurality of target sequences from a plurality of oligonucleotides, wherein each of said polynucleotides shares a plurality of predetermined sequence positions occupied by said oligonucleotides, and wherein each of said polynucleotides comprises a different oligonucleotide in at least one predetermined sequence position, said method comprising:
(a) blocking the 3′ end of each of said oligonucleotides, except for the oligonucleotide comprising the 5′ terminus of each of said polynucleotides, with a blocking group to form a plurality of blocked oligonucleotides;
(b) coupling the 5′ end of each of said blocked oligonucleotides with the 3′ end of a further oligonucleotide of said plurality of oligonucleotides to form a plurality of coupled oligonucleotides, wherein said further oligonucleotide comprises a portion of at least one of said polynucleotides immediately 5′ to said sequence of said blocked oligonucleotide, and wherein each of said coupled oligonucleotides shares at least one oligonucleotide with another coupled oligonucleotide; and
(c) assembling said library of polynucleotides by extension of said coupled oligonucleotides.

41. The method of claim 40 wherein said polynucleotide is DNA, RNA, or DNA/RNA hybrid.

42. The method of claim 40 wherein said oligonucleotides comprise from about 10 to about 200 nucleotides.

43. The method of claim 40 wherein said blocking group comprises solid support.

44. The method of claim 43 wherein said solid support is selected from the group consisting of agarose, polyacrylamide, magnetic beads, polystyrene, polyacrylate, controlled-pore glass, hydroxyethylmethacrylate, polyamide, polyethylene, polyethyleneoxy, and polyethyleneoxy/polystyrene copolymer.

45. The method of claim 40 wherein said blocking group is ddUTP-biotin.

46. The method of claim 40 wherein said coupling is carried out using ligase.

47. The method of claim 46 wherein said ligase is T4 RNA ligase.

48. The method of claim 40 wherein said plurality of oligonucleotides is derived from a parent set of polynucleotides having at least one common property.

49. The method of claim 48 wherein said common property is sequence homology.

50. The method of claim 48 wherein said common property is enzyme activity.

51. The method of claim 48 wherein said common property is ligand binding.

52. The method of claim 48 wherein said set of polynucleotides is optimized.

53. A method of identifying a polynucleotide with a predetermined property, said method comprising, generating a library of polynucleotides according to the method of claim 26, and selecting at least one polynucleotide within said library having said predetermined property.

54. A method of identifying a polynucleotide with a predetermined property, said method comprising, generating a library of polynucleotides according to the method of claim 40, and selecting at least one polynucleotide within said library having said predetermined property.

55. A method of identifying a polynucleotide with a predetermined property, comprising:
(a) generating a library of polynucleotides according to the method of claim 26;
(b) selecting at least one polynucleotide within said library having said predetermined property; and
(c) repeating steps (a) and (b) wherein at least one oligonucleotide of said selected polynucleotides is preferentially incorporated into said library.

56. A method of identifying a polynucleotide with a predetermined property, comprising:
(a) generating a library of polynucleotides according to the method of claim 40;
(b) selecting at least one polynucleotide within said library having said predetermined property; and
(c) repeating steps (a) and (b) wherein at least one oligonucleotide of said selected polynucleotides is preferentially incorporated into said library.


SEQUENCE LISTING (85 sequences)

SEQ ID NO: 1 - protein, 377 aa, Bacillus alcalophilus
MKKPLGKIVASTALLISVAFSSSIASAAEEAKEKYLIGFNEQEAVSEFVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPNAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 271 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 3 - protein, 269 aa, Bacillus sp. KSM-K16
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFQYGAGLDIVAPGVNVQSTYPGSTYASLNGTSMATPHVAGVAAALVKQKNPSWSNVQIRNHLKNTATGLGNTNLYGSGLVNAEAATR

SEQ ID NO: 4 - protein, 269 aa, Bacillus lentus
  UNSURE (215)..(215): X is unknown
AQSVPWGISRVQAPAAHNRGLTGSGVKVAVLDTGISTHPDLNIRGGASFVPGEPSTQDGNGHGTHVAGTIAALDNSIGVLGVAPSAELYAVKVLGASGSGAISSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTXMATPHVAGAAALVKQKNPSWSNVQIRNHLKNTATSLGSTNLYGSGLVNAEAATR

SEQ ID NO: 5 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 6 - protein, 379 aa, Bacillus sp.
MNKKMGKIVAGTALIISVAFSSSIAQAAEEAKEKYLIGFKEQEVMSQFVDQIDGDEYSISSSQVEDVEIDLLHEFDFIPVLSVELDPQDVEALELDPAISYIEEDAEVTTMQTVPWGINRVQAPIAQSRGFTGTGVRVAVLDTGISNHADLRIRGGASFVPGEPNISDGNGHGTHVAGTIAALNNSIGVLGVAPNVDLYGVKVLGASGSGSISGIAQGLQWAANNGMHIANMSLGSSSAGSATMEQAVNQATASGVLVVAASGNSGAGNVGFPARYANAMAVGATDQNNNRASFSQYGAGLDIVAPGVGVQSTVPGNGYSSFNGTSMATPHVAVGAALVKQKNPSWSNVQIRNHLKNTATNLGNTNQFSGLVNAEAATR

SEQ ID NO: 7 - protein, 382 aa, Bacillus sp.
MKKLFTKVVASAALLLSISLTATSVSAEEQKKQYLIGFENQLQVTEFVESSDKGQSEMSLFAEVNDESIEMELLYEFEDIPVVSVELSPEDVKDLEKDPSITYIEEDIEVTITNQVTPWGITRVQAPTAWTRGYTGTGVRVAVLDTGISTHPDLNIRGGVSFVPGEPSYQDGNGHGTHVAGTIAALNNSIGVVGVAPNAELYAVKVLGANGSGSVSSIAQGLQWTAQNNIHVANLSLGSPVGSQTLELAVNQATNAGVLVVAATGNNGSGTVSYPARYANALAVGATDQNNNRASFSQYGTGLNIVAPGVGIQSTYPGNRYASLSGTSMATPHVAGVAALVKQKNPSWSNTQIRQHLTSTATSLGNSNQFGSGLVNAEAATR

SEQ ID NO: 8 - protein, 375 aa, Bacillus sp.
MNLQKIRSALKVKQSALVSSLTILFLIMLVGTTSANGAKQEYLIGFNSDKAKGLIQNAGGEIHHEYTEFPVIYAELPEAAVSGLKNNPHIDFIEENEEVEIAQTVPWGIPYIYSDVVHRQGYFGNGVKVAVLDTGVAPHPDLHIRGGVASFISTENTYVDYNGHGTHVAGTVAALNNSYGVLGVAPGAELYAVKVLDRNGSGSHASIAQGIEWAMNNGMDIANMSLGSPSGSTTLQLAADRARNAGVLLIGAAGNSGQQGGSNNMGYPARYASVMAVGAVDQNGNRANFSSYGSELEIMAPGVNINSTYLNNGYRSLNGTSMASPHVAGVAALVKQKHPHLTAAQIRNRMNQTAIPLGNSTYYGNGLVDAEYAAQ

SEQ ID NO: 9 - protein, 372 aa, Bacillus licheniformis
MMRKKSFWLGMLTAFMLVFTMAFSDSASAAQPAKNVEKDYIVGFKSGVKTASVKKDIIKESGGKVDKQFRIINAAKAKLDEALKEVKNDPVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINV

SEQ ID NO: 10 - protein, 379 aa, Bacillus licheniformis
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGTYSGIVSGIEWATTNGMDVINMSLGGPSGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 11 - protein, 379 aa, Bacillus licheniformis
MMRKKSFWLGMLTALMLVFTMAFSDSASAAQPAKNVEKDYIVGFKSGVKTASVKKDIIKESGGKVDKQFRIINAAKAKLDKEALEEVKNDPDVAYVEEDHVAHALAQTVPYGIPLIKADKVQAQGYKGANVKVAVLDTGIQASHPDLNVVGGASFVAGEAYNTDGNGHGTHVAGTVAALDNTTGVLGVAPNVSLYAVKVLNSSGSGSYSGIVSGIEWATTNGMDVINMSLGGPSGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 12 - protein, 379 aa, Bacillus licheniformis
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVGHGLGQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWVTTNGMDVINMSLGGASGSTAMKQAVDNAYARGVVVVAAAGNSGSSGNTNTIGYPAKCDSVIPVGGEDSNSNRSSFSSVGAELEVMAPVSGVYSTYPTNTYTTLNGTSMASPHVAGTSALILSKHPNLSASQVRNRLSRTATYLGSSFYYGKGLINVEAAAQ

SEQ ID NO: 13 - protein, 379 aa, Bacillus licheniformis
MMRKKSFWLGMLTALMLVFTMAFSDSASAAQPGKNVEKDYIVGFKSGVKTASVKKDIIKESGGKVDKQFRIINAGKAKLDKEALKEVKNDPDVAYVEEDHVAHVLGQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSAIVSGIEWATTTGMDVINMSLGGASVSTAMKQAVDHAYARGAVVVSSAGNSGSSGNTNTIGYPAKYDSVIAVGAVDSNSNRASFSSVGAELEVMAPGAGVYSTYPTNTYATLNGTSMASPHVAGAAALILSKHPNLSASQVRTRLSRTATYLGSSFSYGRGLINVEAAAQ

SEQ ID NO: 14 - protein, 378 aa, Bacillus licheniformis
MMRKKSFWFGMLTAFMLVFTMEFSDSASAAQPGKNVEKDYFVGFKSGVKTASVKKDIIKESGGKVDKQFRIINAAKATLDKEALKEVKNDPDVAYVEEDHVAHALGQTVPYGIPLIKADKVQAQGFKGANVKVAVLDTGIQASHPDLNVVGGASFVAGEAYNTDGNGHGTHVAGTVAALDNTTGLGVAPSVSLFAVKVLNSSGSGSYSGIVSGIEWATTNGMDVINMSLGGPSGSTAMKQAVDNAYSKGVVPVAAAGNSGSSGYTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 15 - protein, 310 aa, Bacillus licheniformis
RIINAAKAKLDKEALEEVKNDPDVAYVEEDHVAHALAQTVPYGIPLIKADKVQAQGYKGANVKVAVLDTGIQASHPDLNVVGGASFVAGEAYNTDGNGHGTHVAGTVAALDNTTGVLGVAPNVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 16 - protein, 380 aa, Bacillus subtilis var. natto
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSVGSELDVMAPGVSIQSSTLPGGTYGAYNGTSMATHVAGAAALILSHPTWTNAQVRDRLESTATYLGNSFYYGKGLINVQAAAQ

SEQ ID NO: 17 - protein, 274 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTCMATPHVAGAAALILSKHPTWTNAQVRDRLESTATYLGNSFYYGKGLINVQAAAQ

SEQ ID NO: 18 - protein, 275 aa, Bacillus pumilus
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSANQRASFSSAGSELDVMAPGVSIQSTLPGGTYGAYNGTSMATPHVAGAAALILSKHPTWTNAQVRDRLESTATYLGSSFYYGKGLINVQAAAQ

SEQ ID NO: 19 - protein, 380 aa, Bacillus amyloliquefaciens
MRGKKVWISLLFALALIFTMAFGSTSSAQAAGKSNGEKKYIVGFQTMSTMSAAKKKDVISEKGGKVQKQFKYVDAASATLNEKAVKELKKDPSVAYVEEDHVAHAYAQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAVDKAVASGVVVVAAAGNEGTSGSSSTVGYPGKYPSVIAVGAVDSSNQRASFSSVGPELDVMAPGVSIQSTLPGNKYGAYNGTSMASPHVAGAAALILSKHPNWTNTQVRSSLENTTTKLGDSFYYGKGLINVQAAAQ

SEQ ID NO: 20 - protein, 382 aa, Bacillus amyloliquefaciens
MISLLFALALIFTMAFGSTSSAQAAGKSNGEKKYIVGFKQTMSTMSAAKKKDVISEKGGKVQKQFKYVDAASATLNEKAVKELKKDPSVAYVEEDHVAHAYAQSVPYGVSQIKAPALHSQGYTGSNVKVAVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVDKAVASGVVVVAAAGNEGTSGSSSTVGYPGKYPSVIAVGAVDSSNQRASFSSVGPELDVMAPGVSIQSTLPGNKYGAYNGTSMASPHVAGAAALILSKHPNWTNTQVRSSLENTTTKLGDSFYYGKGLINVQAAAQ

SEQ ID NO: 21 - protein, 279 aa, Bacillus amyloliquefaciens
AQSVPYGVSQIKAPALHSQGYTGSNVKVAVIDSGIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGAYSGTCMASPHVAGAAALILSKHPNWTNTQVRSSLENTTTKLGDSFYYGKGLINVQAAAQ

SEQ ID NO: 22 - protein, 267 aa, Bacillus amyloliquefaciens
AQSVPYGVSQIKAPALSHSQGYTGSNVKVAVIDSGIDSSHPDLKVAGGASFVPSETNPFQDNNSHGTHVAGTVAAVAPSASLYAVKVLGADGSGQYSWIINGIEWAIANNMDVINMSLGGSPGSAALKAAVDKAVASGVVVVAAAGNEGTSGSSSTVGYPGKYPSVIAVGAVDSSNQRASFSSVGPELDVMAPGVSIQSTLPFNKYGAKSGTCMASPHVAGAAALILSKHPNWTNTQVRSSLENTTTKLGDSFYYGKGLINVQAAAQ

SEQ ID NO: 23 - protein, 275 aa, Bacillus amyloliquefaciens
AQSVPYGVSQIKAPALHSQGYCGSNVKVAVIDSGIDSSHPDLKVAGGASFVPSETNPFQDNNSHGTHVAGTVAALNNSIGVLGVAPCASLYAVKVLGADGSGQYSWIINGIEWAIANNMDVINMSLGGPSGSAALKAAVDKAVASGVVVVAAAGNEGTSGSSSTVGYPAKYPSVIAVGAVDSSNQRASFSSVGPELDVMAPGVSICSTLPGNKYGAKSGTSMASPHVAGAAALILSKHPNWTNTQVRSSLENTTTKLGDSFYYGKGLINVQAAAQ

SEQ ID NO: 24 - protein, 371 aa, Bacillus halodurans
MKRRVHLIMTILMIVLATGTAFADNREDTEDTEEYLVGFKNEAAVQAFSNNVTTSAVEVQHEYENLPVIVSELSTEVAQLLENDPSVEFIEKNERVYLDPLVMNNIQETDIPKLEERMKRGDGVKIAVLDTGIASHDDLHVIDGVSFVSVEPFYRDLNGHGTHVAGTIAAQENDEASTGIAPNVELYAVKVLNGLGAGSIASITNGVDWAISNDMDIINMSLGTNTDSEALQRAVERAHDHGILIIAAAGNSGEADKQHTIDYPARYDSVVAVGAVDGNNERASFSSYGEQLEIMAPGVEIHSTFLFNRYERLSGTSMASPHVTGAAALIKSNNPELTNEQIRKRLNTTATPLGNPFYYGHGLLNVDAALD

SEQ ID NO: 25 - protein, 435 aa, Bacillus smithii
MKKFKRKGRLLDEKHEMAIFFLFLLVILPFKAYGAESDEYMILFRHHIDDGILKKYDIHAQKKYETIPTVLAELNKSQVDKLRSEPEIAKVQANKTYKVQSQIETWGYKKIYEDSQYRSPFTGKGVKVAVIDTGIASNHPDLKVKGGTCVIRSDCGKGYNDDNGHTHVAGIIGALDNGVGIVGVAPDADLYAVKAFDEFGEGSTSSITAGVDWAIQHHMDIINLSVTTVSDDPVLKSALDKAYNAGILITAAANDGDSVGSKNTILYPAKYSSVIAVGSVDSRLQRLPFSATGPELEIVAPGQYVFSTFPINLDTTDGKKDGYTALSGTSMALPHLYTGALAATLKTSIKTNRPAGNPQNTSDQNAKDLGTAGKDSLYGYGLVQIKTFQPTLSSDMAVKAVKADNGLWSSRSISPIPFKSLAEKKRRNGGRHYKQ

SEQ ID NO: 26 - protein, 648 aa, Bacillus subtilis
MKNMSCKLVVSVTLFFSFLTIGPLAHAQNSSEKEVIVVYKNKAGKETILDSDADVEQQYKHLPAVAVTADQETVKELKQDPDILYVENNVSFTAADSTDFKVLSDGTDTSDNFEQWNLEPIQVKQAWKAGLTGKNIKIAVIDSGISPHDDLSIAGGYSAVSYTSSYKDDNGHGTHVAGIIGAKHNGYGIDGIAPEAQIYAVKALDQNGSGDLQSLLQGIDWSIANRMDIVNMSLGTTSDSKILHDAVNKAYEQGVLLVAASGNDGNGKPVNYPAAYSSVVAVSATNEKNQLASFSTTGDEVEFSAPGTNITSTYLNQYYATGSGTSQATPHAAAMFALLKQRDPAETNVQLREEMRKNIVDLGTAGRDQQFGYGLIQYKAQATDSAYAAAEQAVKKAEQTKAQIDINKARELISQLPNSDAKTALHKRLDKVQSYRNVKDAKDKVAKAEKYKTQQTVDTAQTAINKLPNGTDKKNKQKRLDQVKRYIASKQAKDKVAKAEKSKKKTDVDSAQSAIGKLPASSEKTSLQKRLNKVKSTNTAQQSVSAAEKKSTDANAAKAQSAVNQSAVNQLQAGKDKTALQKRLDKVKKKVAAAEAKKVETAKAKVKKAEKDKTKKSKTSAQSAVNQLKASNEKTKLQKRLNAVKPKK

SEQ ID NO: 27 - protein, 440 aa, Aeropyrum pernix
MVAVVTGVIQVGTKIAAIAIALIFILPLFPVYTGSAAGASTVVIAKINPEEFNPKAVEALQGKVIYVADLAPVAIISIPGKAVGLLSKLPGVVSVSEDGVVQAMAKPPWAGGGNKSQPAEVLPWGVDYIDAELVWPDGVTGWVDVNGDGDGEIEVAVIDTGVDKDHPDLAGNIVWGISVLNGRISSNYQDRNGHGTHVTGTVAAIDNDIGVIGVAHSVEIYAVKALGNGGYGSWSDLIIAIDLAVKGPDGVIDADGDGVVAGDPDDDAPEVISMSLGGSSPPPELHDVIKAAYNLGITIVAAAGNDGADSPSYPAAYPEVIAVGAIDENGNVPSWSNRNPEVAAPGVNILSTYPDDTYEELSGTSMATPHVSGTVALIQAARLAAGLPLLPPGSESDTTPDTVRGVLHTTATDAGDPGYDSLYGYGIIDAYDAVQTAVSS

SEQ ID NO: 28 - protein, 554 aa, Bacillus halodurans
MKTMKTLVIVIGVLALLFIVAIGHVRDQDQALIQTKEIPKTLETEAMDHLLAEDLSLTTSMFIKQMAEQLQRWSEQLEEDPTIKDEFRQQIDEHPHMQFAIAEANKITQKVGTLHRDDVKALTHVHHNQRYSDPYNVDDSTYMLIGESTDDGRLLIGELNLEFVKKYVKDIAAVADTNGNFFIGGDNPDVSWQDQDERATQLTSETVPELGWDIHVQSEGQEEEGPAYHEHQAVIRFKPNRDPAAWFATNPYRVVEEAPPFFVIESPNQTTVEIVEALSRDYDLDFAEPNYRFTKQIQAPVTPNDEFFKEYQWNLQQIDIEEGWSLASGENVKIAILDTGVDPNHPDIKDKIVNGYNAVEGNNNFADKHGHGTHVAGVAAAVTNNVTGIAGISWKSEILPVKVLNDNGEGSSFEVAKGIYWATDHGAKVINMSLGDYYHSDALRDAVKYAYDHDVVLIAASGNDNVEDPLYPAIYEEVLTVAAVDDTRNRAFFSNFGKHIDVTAPGEHIPDLSNQEVMDIMKKTAKDLGPKGHDVYYGHGEIDIEAALKAIRTS

SEQ ID NO: 29 - protein, 782 aa, Bacillus halodurans
MNKRLKRWSAIMSIVLLVNLLVPVGTFADTDEEEKVDIIVTYKDVVSESPDSRLSVYENEQTMKTLPIKTMTVPVSEVERLKEDPNVVSVSLDQPLQLMSDTRELGEHDWNNDMVKAFDAWDDGYTGKGVKVAVFDTGFDGHQDITYAGGHSVFEGEPYTHDHHGHGTHVAGIIAGAREGTLHQGIAPDVQLYGVKVFSQEKGGNTSDLIAGIDWAIQEGMDIINMSLGYTNEVPAVHTAIKQAVAQEILVVAASGNGGKADGSGETIEYPAKYDEVIAVASVDKEMKRTNTSATGVENELAAPGHLIGGLAPGNKYVFMSGTSQATPHVTSLAAIIMGKHPELSSQQIRALLVEQSLDLGSEGHDRLYGYGLAQYVSSTPPDEEENEESPAENPQEQPSDGKENEGSEDQGSTPPDEEENEESPAEDPQEQPSDGKENKGSENQGSTPPDEEENEESPAEVPQEQPSDGKENEGSEDQGSTPPDEEENEESPAEDPQEQPSDKENEESKNPDSAPPAGEKKEGKQTARVQVKPVNLGGVAIVSNADVASVLDNNGTLVVFFDSALDDLTRLALTADQVKELKDRGITLVIAKHDELVIPHGVFKAGDVVIEFERVVGKGIPYAGQAKSTVYQFKIIQNGQQVRHFDEEIEMGFRVDQEKNVNNLKIYYWNESLNEWEKIGGNYQEGFIVARTNFEETKSGTPISGGKTNGNSTTEGTTNRGTSKNGTGSEPQAEESNNEQNNKDGTLPKTATNLYNSLAIGALLLLIGFVLLRKSKRRIVE

SEQ ID NO: 30 - DNA, 100 bp, Artificial
  Novel Sequence
agaggatccccgggtaccggtagaaaaaatgagtaaaggagaagaacttttcactggagttgtcccaattcttgttgaattagatggtgatgttaatggg

SEQ ID NO: 31 - DNA, 60 bp, Artificial
  Novel Sequence
cacaaattttctgtcagtggagagggtgaaggtgatgcaacatacggaaaacttaccctt

SEQ ID NO: 32 - DNA, 60 bp, Artificial
  Novel Sequence
aaatttatttgcactactggaaaactaccggttccatggccaacacttgtcactactttc

SEQ ID NO: 33 - DNA, 100 bp, Artificial
  Novel Sequence
tcttatggtgttcaatgcttttcaagatacccagatcatatgaaacggcatgactttttcaagagtgccatgcccgaaggttatgtacaggaaagaacta

SEQ ID NO: 34 - DNA, 20 bp, Artificial
  Novel Sequence
agaggatccccgggtaccgg

SEQ ID NO: 35 - DNA, 20 bp, Artificial
  Novel Sequence
aagggtaagttttccgtatg

SEQ ID NO: 36 - DNA, 20 bp, Artificial
  Novel Sequence
gaaagtagtgacaagtgttg

SEQ ID NO: 37 - DNA, 20 bp, Artificial
  Novel Sequence
tagttctttcctgtacataa

SEQ ID NO: 38 - DNA, 100 bp, Artificial
  Novel Sequence
agaggatccccgggtaccggtagaaaaaatgaggtcttccaagaatgttatcaaggagttcatgaggtttaaggttcgcatggaaggaacggtcaatggg

SEQ ID NO: 39 - DNA, 60 bp, Artificial
  Novel Sequence
cacgagtttgaaatagaaggcgaaggagaggggaggccatacgaaggccacaataccgta

SEQ ID NO: 40 - DNA, 63 bp, Artificial
  Novel Sequence
aagcttaaggtaaccaaggggggacctttgccatttgcttgggatattttgtcaccacaattt

SEQ ID NO: 41 - DNA, 93 bp, Artificial
  Novel Sequence
cagtatggaagcaaggtatatgtcaagcaccctgccgacataccagactataaaaagctgtcatttcctgaaggatttgtacaggaaagggtc

SEQ ID NO: 42 - DNA, 18 bp, Artificial
  Novel Sequence
tacggtattgtggccttc

SEQ ID NO: 43 - DNA, 21 bp, Artificial
  Novel Sequence
aaattgtggtgacaaaatatc

SEQ ID NO: 44 - DNA, 20 bp, Artificial
  Novel Sequence
gaccctttcctgtacaaatc

SEQ ID NO: 45 - DNA, 60 bp, Artificial
  Novel Sequence
atgatgaggaaaaagagtytttggtttgggatgttgacggccyttatgctcgtgttcacg

SEQ ID NO: 46 - DNA, 60 bp, Artificial
  Novel Sequence
atggmgttcagcgattccgcttctgctgctcaaccggsgaaaaatgttgaaaaggattat

SEQ ID NO: 47 - DNA, 60 bp, Artificial
  Novel Sequence
wttgtcggatttaagtcaggagtgaaaaccgcatctgtcaaaaaggacatcatcaaagag

SEQ ID NO: 48 - DNA, 60 bp, Artificial
  Novel Sequence
agcggcggaaaagtggacaagcagtttagaatcatcaacgcggsaaaagcgacgctagac

SEQ ID NO: 49 - DNA, 60 bp, Artificial
  Novel Sequence
aaagaagcgcttraggaagtcaaaaatgatccggatgtcgcttatgtggaagaggatcat

SEQ ID NO: 50 - DNA, 60 bp, Artificial
  Novel Sequence
gtggsacatgbgttggsacaaaccgttccttacggcattcctctcattaaagcggacaaa

SEQ ID NO: 51 - DNA, 60 bp, Artificial
  Novel Sequence
gtgcaggctcaaggctwtaagggagcgaatgtaaaagtagccgtcctggatacaggaatc

SEQ ID NO: 52 - DNA, 60 bp, Artificial
  Novel Sequence
caagcttctcatccggacttgaacgtagtcggcggagcaagctttgtggctggcgaagct

SEQ ID NO: 53 - DNA, 60 bp, Artificial
  Novel Sequence
tataacaccgacggcaacggacacggcacacatgttgccggtacagtagctgcgcttgac

SEQ ID NO: 54 - DNA, 60 bp, Artificial
  Novel Sequence
aatacaacgggtgtattaggcgttgcgccaartgtatccttgawtgcggttaaagtactg

SEQ ID NO: 55 - DNA, 60 bp, Artificial
  Novel Sequence
aattcaagcggaagcggaasttacagcgsaattgtaagcggaatcgagtgggygacaaca

SEQ ID NO: 56 - DNA, 60 bp, Artificial
  Novel Sequence
amtggcatggatgttatcaatatgagccttgggggascatcagkgtcgacagcgatgaaa

SEQ ID NO: 57 - DNA, 60 bp, Artificial
  Novel Sequence
caggcagtcgacmatgcatatkctargggggytgtcsytgtakctkctgcagggaacagc

SEQ ID NO: 58 - DNA, 60 bp, Artificial
  Novel Sequence
ggatcttcaggawatacgaatacaattggctatcctgcgaaatrtgattctgtcatcscg

SEQ ID NO: 59 - DNA, 60 bp, Artificial
  Novel Sequence
gttggtgsggwggactctaacagcaacagakcgtcattttccagtgtgggagcagagctt

SEQ ID NO: 60 - DNA, 60 bp, Artificial
  Novel Sequence
gaagtcatggctcctgkgkcgggcgtatacagcacttacccaacgartacttatrcgaca

SEQ ID NO: 61 - DNA, 60 bp, Artificial
  Novel Sequence
ttgaacggaacgtcaatggcttctcctcatgtagcgggarcgkcggctttgatcttgtca

SEQ ID NO: 62 - DNA, 60 bp, Artificial
  Novel Sequence
aaacatccgaacctttcagcttcacaagtccgcamtcgtctctccagkacggcgacttat

SEQ ID NO: 63 - DNA, 60 bp, Artificial
  Novel Sequence
ttgggaagctccttctmttatgggargggtctgatcaatgtcgaagctgccgctcaataa

SEQ ID NO: 64 - DNA, 57 bp, Artificial
  Novel Sequence
atgagaggcaaaaaagtatggatcagtttgctgtttgctttagcgttaatctttacg

SEQ ID NO: 65 - DNA, 39 bp, Artificial
  Novel Sequence
atgatcagtttgctgtttgctttagcgttaatctttacg

SEQ ID NO: 66 - DNA, 63 bp, Artificial
  Novel Sequence
atggcgttcggcagcacatcctctgcccaggcggcagggaaatcaaacggggaaaagaaatat

SEQ ID NO: 67 - DNA, 66 bp, Artificial
  Novel Sequence
attgtcgggtttaaacagacaatgagcacgatgagcgccgctaagaagaaagatgtcatttctgaa

SEQ ID NO: 68 - DNA, 60 bp, Artificial
  Novel Sequence
aaaggcgggaaagtgcaaaagcaattcaaatatgtagacgcagcttcagctacattaaac

SEQ ID NO: 69 - DNA, 57 bp, Artificial
  Novel Sequence
gaaaaagctgtaaaagaattgaaaaaagacccgagcgtcgcttacgttgaagaagat

SEQ ID NO: 70 - DNA, 63 bp, Artificial
  Novel Sequence
cacgtagcacatgcgtacgcgcagtccgtgccttacggcgtatcacaaattaaagcccctgct

SEQ ID NO: 71 - DNA, 60 bp, Artificial
  Novel Sequence
ctgcactctcaaggctacwstggatcaaatgttaaagtagcggttatcgacagcggtatc

SEQ ID NO: 72 - DNA, 60 bp, Artificial
  Novel Sequence
gattcttctcatcctgatttaaaggtagcaggcggagccagcwtkgttccttctgaaaca

SEQ ID NO: 73 - DNA, 63 bp, Artificial
  Novel Sequence
aatcctttccaagacaacaactctcacggaactcacgttgccggcacagttgcggctcttaat

SEQ ID NO: 74 - DNA, 60 bp, Artificial
  Novel Sequence
aatcctttccaagacaacaactctcacggaactcacgttgccggcacagttgcggctgtt

SEQ ID NO: 75 - DNA, 60 bp, Artificial
  Novel Sequence
aactcaatcggtgtattaggcgttgcgccawgtgcatcactttacgctgtaaaagttctc

SEQ ID NO: 76 - DNA, 36 bp, Artificial
  Novel Sequence
gcgccatcagcatcactttacgctgtaaaagttctc

SEQ ID NO: 77 - DNA, 60 bp, Artificial
  Novel Sequence
ggtgctgacggttccggccaatacagctggatcattaacggaatcgagtgggcgatcgca

SEQ ID NO: 78 - DNA, 60 bp, Artificial
  Novel Sequence
aacaatatggacgttattaacatgagcctcggcggaccttctggttctgctgctttaaaa

SEQ ID NO: 79 - DNA, 60 bp, Artificial
  Novel Sequence
gcggcagttgataaagccgttgcatccggcgtcgtagtcgttgcggcagccggtaacgaa

SEQ ID NO: 80 - DNA, 60 bp, Artificial
  Novel Sequence
ggcacttccggcagctcaagcacagtgggctaccctgsgaaatacccttctgtcattgca

SEQ ID NO: 81 - DNA, 60 bp, Artificial
  Novel Sequence
gtaggcgctgttgacagcagcaaccaaagagcatctttctcaagcgtaggacctgagctt

SEQ ID NO: 82 - DNA, 60 bp, Artificial
  Novel Sequence
gatgtcatggcacctggcgtatctatcyrkagcacgcttcctggaaacaaatacggggcg

SEQ ID NO: 83 - DNA, 60 bp, Artificial
  Novel Sequence
wakartggtacgtcaatggcatctccgcacgttgccggagcggctgctttgattctttct

SEQ ID NO: 84 - DNA, 60 bp, Artificial
  Novel Sequence
aagcacccgaactggacaaacactcaagtccgcagcagtttagaaaacaccactacaaaa

SEQ ID NO: 85 - DNA, 60 bp, Artificial
  Novel Sequence
cttggtgattctttctactatggaaaagggctgatcaacgtacaggcggcagctcagtaa